(19) United States
(12) Patent Application Publication
(10) Pub. No.: US 2006/0024357 A1
(43) Pub. Date: Feb. 2, 2006

(54) Wound healing polymer compositions and methods for use thereof

(75) Inventors: Kenneth W. Carpenter, San Diego, CA (US); Huashi Zhang, San Diego, CA (US); Brendan J. McCarthy, Cardiff, CA (US); Istvan Szinai, San Diego, CA (US); William G. Turnell, San Diego, CA (US); Sindhu M. Gopalan, San Diego, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: MEDIVAS, LLC, San Diego, CA (US)

(21) Appl. No.: 11/128,903

(22) Filed: May 12, 2005

Related U.S. Application Data
(60) Provisional application No. 60/570,668, filed on May 12, 2004.
(60) Provisional application No. 60/605,381, filed on Aug. 27, 2004.

(51) Int. Cl. A61K 9/14; A61L 15/00

(52) U.S. Cl. 424/445; 424/486; 424/93.7

(57) ABSTRACT

The present invention provides bioactive polymer compositions that can be formulated to release a wound healing agent at a controlled rate by adjusting the various components of the composition. The composition can be used in an external wound dressing, as a polymer implant for delivery of the wound healing agent to an internal body site, or as a coating on the surface of an implantable surgical device to deliver wound healing agents that are covalently attached to a biocompatible, biodegradable polymer and/or embedded within a hydrogel. Methods of using the invention bioactive polymer compositions to promote natural healing of wounds, especially chronic wounds, are also provided. Examples of biodegradable copolymer polyesters useful in forming the blood-compatible, hydrophilic layer or coating include copolyester amides, copolyester urethanes, glycolide-lactide copolymers, glycolide-caprolactone copolymers, poly-3-hydroxy butyrate-valerate copolymers, and copolymers of the cyclic diester monomer, 3-(S)[(alkyloxycarbonyl)methyl]-1,4-dioxane-2,5-dione, with L-lactide. The glycolide-lactide copolymers include poly(glycolide-L-lactide) copolymers formed utilizing a monomer mole ratio of glycolic acid to L-lactic acid ranging from 5:95 to 95:5 and preferably a monomer mole ratio of glycolic acid to L-lactic acid ranging from 45:65 to 95:5. The glycolide-caprolactone copolymers include glycolide and ε-caprolactone block copolymer, e.g., Monocryl or Poliglecaprone.

### RELATED APPLICATION

This application relies for priority under 35 U.S.C. 119(e) on U.S. provisional application Nos. 60/570,668, filed May 12, 2004, and 60/605,381, filed Aug. 27, 2004, the content of each of which is incorporated herein by reference in its entirety.

### FIELD OF THE INVENTION

The invention relates generally to compositions used in wound healing, and in particular to biodegradable polymer compositions that promote healing at wound sites.

### BACKGROUND INFORMATION

The normal endothelium, which lines blood vessels, is uniquely and completely compatible with blood. Endothelial cells initiate metabolic processes, like the secretion of prostacylin and endothelium-derived relaxing factor (EDRF), which actively discourage platelet deposition and thrombus formation in vessel walls. However, damaged arterial surfaces within the vascular system are highly susceptible to thrombus formation. Abnormal platelet deposition, resulting in thrombosis, is more likely to occur in vessels in which endothelial, medial and adventitial damage has occurred. While systemic drugs have been used to prevent coagulation and to inhibit platelet aggregation, a need exists for a means by which a damaged vessel can be treated directly to prevent thrombus formation and subsequent intimal smooth muscle cell proliferation.

Current treatment regimes for stenosis or occluded vessels include mechanical interventions. However, these techniques also serve to exacerbate the injury, precipitating new smooth muscle cell proliferation and neointimal growth. For example, stenotic arteries are often treated with balloon angioplasty, which involves the mechanical dilation of a vessel with an inflatable catheter. The effectiveness of this procedure is limited in some patients because the treatment itself damages the vessel, thereby inducing proliferation of smooth muscle cells and reocclusion or restenosis of the vessel. It has been estimated that approximately 30 to 40 percent of patients treated by balloon angioplasty and/or stents may experience restenosis within one year of the procedure.

To overcome these problems, numerous approaches have been taken to providing stents useful in the repair of damaged vasculature. In one aspect, the stent itself reduces restenosis in a mechanical way by providing a larger lumen. For example, some stents gradually enlarge over time. To prevent damage to the lumen wall during implantation of the stent, many stents are implanted in a contracted form mounted on a partially expanded balloon of a balloon catheter and then expanded in situ to contact the lumen wall. U.S. Pat. No. 5,059,211 discloses an expandable stent for supporting the interior wall of a coronary artery wherein the stent body is made of a porous bioabsorbable material. To aid in avoiding damage to vasculature during implant of such stents, U.S. Pat. No. 5,662,960 discloses a friction-reducing coating of commingled hydrogel suitable for application to polymeric plastic, rubber or metallic substrates that can be applied to the surface of a stent.

A number of agents that affect cell proliferation have been tested as pharmacological treatments for stenosis and restenosis in an attempt to slow or inhibit proliferation of smooth muscle cells. These agents have included heparin, coumarin, aspirin, fish oils, calcium antagonists, steroids, prostacyclin, ultraviolet irradiation, and others. Such agents may be systemically applied or may be delivered on a more local basis using a drug delivery catheter or a drug eluting stent. In particular, biodegradable polymer matrices loaded with a pharmaceutical may be implanted at a treatment site. As the polymer degrades, a medicament is released directly at the treatment site. The rate at which the drug is delivered is dependent upon the rate at which the polymer matrix is resorbed by the body. U.S. Pat. No. 5,342,348 to Kaplan and U.S. Pat. No. 5,419,760 to Norciso are exemplary of this technology. U.S. Pat. No. 5,766,710 discloses a stent formed of composite biodegradable polymers of different melting temperatures.

Porous stents formed from porous polymers or sintered metal particles or fibers have also been used for release of therapeutic drugs within a damaged vessel, as disclosed in U.S. Pat. No. 5,843,172. However, tissue surrounding a porous stent tends to infiltrate the pores. In certain applications, pores that promote tissue ingrowth are considered to be counterproductive because the growth of neointima can occlude the artery, or other body lumen, into which the stent is being placed.

Delivery of drugs to the damaged arterial wall components has also been explored by using latticed intravascular stents that have been seeded with sheep endothelial cells engineered to secrete a therapeutic protein, such as t-PA (D. A. Dichek et al., Circulation, 80:1347-1353, 1989). However, endothelium is known to be capable of promoting both coagulation and thrombolysis.

Another approach to controlling the healing of a damaged artery or vein is to induce apoptosis in neointimal cells to reduce the size of a stenotic lesion. U.S. Pat. No. 5,776,905 to Gibbons et al. describes induction of apoptosis by administering anti-sense oligonucleotides that counteract the anti-apoptotic gene, bcl-x, which is expressed at high levels by neointimal cells. These anti-sense oligonucleotides are intended to block expression of the anti-apoptotic gene bcl-x so that the neointimal cells are induced to undergo programmed cell death.

Under certain conditions, the body naturally produces another drug, nitric oxide, which has an influence on cell apoptosis among its many effects. As is explained in U.S. Pat. No. 5,759,836 to Amin et al., nitric oxide (NO) is produced by an inducible enzyme, nitric oxide synthase, which belongs to a family of proteins beneficial to arterial homeostasis.

However, the effect of nitric oxide in the regulation of apoptosis is complex. A pro-apoptotic effect seems to be linked to pathophysiological conditions wherein high amounts of NO are produced by the inducible nitric oxide synthase. By contrast, an anti-apoptotic affect results from the continuous, low level release of endothelial NO, which inhibits apoptosis and is believed to contribute to the anti-atherosclerotic function of NO. Dimmeler in “Nitric Oxide and Apoptosis: Another Paradigm for the Double-Edged Role of Nitric Oxide” (Nitric Oxide 1(4):275-281, 1997) discusses the pro- and anti-apoptotic effects of nitric oxide.

To prevent neointimal proliferation that leads to stenosis or restenosis, U.S. Pat. No. 5,766,584 to Edelman et al. describes a method for inhibiting vascular smooth muscle cell proliferation following injury to the endothelial cell lining by creating a matrix containing endothelial cells and surgically wrapping the matrix about the tunica adventitia. The matrix, and especially the endothelial cells attached to the matrix, secretes products that diffuse into surrounding tissue, but do not migrate to the endothelial cell lining of the injured blood vessel.

In a healthy individual in response to endothelial damage, the vascular endothelium participates in many homeostatic mechanisms important for normal wound healing, the regulation of vascular tone and the prevention of thrombosis. A primary mediator of these functions is endothelium-derived relaxing factor (EDRF). First described in 1980 by Furchgott and Zawadzki (Furchgott and Zawadzki, Nature (Lond.) 288:373-376, 1980) EDRF is either nitric oxide (Moncada et al., Pharmacol Rev. 43:109-142, 1991.) (NO) or a closely related NO-containing molecule (Myers et al., Nature (Lond.), 345:161-163, 1990).

Removal or damage to the endothelium is a potent stimulus for neointimal proliferation, a common mechanism underlying the restenosis of atherosclerotic vessels after balloon angioplasty. (Liu et al., Circulation, 79:1374-1387, 1989); (Fems et al., Science, 253:1129-1132, 1991). Stent-induced restenosis is caused by local wounding of the luminal wall of the artery. Further, restenosis is the result of a chronically-stimulated wound-healing cycle.

The natural process of wound healing involves a two-phase cycle: blood coagulation and inflammation at the site of the wound. In healthy individuals, these two cycles are counterbalanced, each including a natural negative feedback mechanism that prevents over-stimulation. For example, in the coagulation enzyme pathway thrombin factor Xa operates upon factor VII to control thrombus formation and, at the same time stimulates production of PARs (Protease Activated Receptors) by pro-inflammatory monocytes and macrophages. Nitric oxide produced endogenously by endothelial cells regulates invasion of the proinflammatory monocytes and macrophages. In the lumen of an artery, this two-phase cycle results in influx and proliferation of healing cells through a break in the endothelium. Stabilization of the vascular smooth muscle cell population by this natural two-phase counterbalanced process is required to prevent neointimal proliferation leading to restenosis. The absence or scarcity of endogenously produced nitric oxide caused by damage to the endothelial layer in the vasculature is thought to be responsible for the proliferation of vascular smooth muscle cells. This situation results in restenosis following vessel injury, for example following angioplasty.

Nitric oxide dilates blood vessels (Vallance et al., Lancet, 2:997-1000, 1989), inhibits platelet activation and adhesion (Radomski et al., Br. J Pharmacol, 92:181-187, 1987) and, in vitro, nitric oxide limits the proliferation of vascular smooth muscle cells (Garg et al., J. Clin. Invest. 83:1774-1777, 1986). Similarly, in animal models, suppression of platelet-derived mitogens by nitric oxide decreases intimal proliferation (Fems et al., Science, 253:1129-1132, 1991). The potential importance of endothelium-derived nitric oxide in the control of arterial remodeling after injury is further supported by recent preliminary reports in humans suggesting that systemic NO donors reduce angiographic-restenosis six months after balloon angioplasty (The ACCORD Study Investigators, J. Am. Coll. Cardiol. 23:59A. (Abstr.), 1994).

Damage to the endothelial and medial layers of a blood vessel, such as often occurs in the course of balloon angioplasty and stent procedures, has been found to stimulate neointimal proliferation, leading to restenosis of atherosclerotic vessels.

The earliest understanding of the function of the endothelium within an artery was its action as a barrier between highly reactive, blood borne materials and the intima of the artery. A wide variety of biological activity within the artery wall is generated when platelets, monocytes and neutrophils infiltrate intima. These reactions result from release of activating factors such as ATP and PDGF from platelets and IL-1, IL-6, TNFa and bFGF from monocytes and neutrophils. An important consequence of release of these activating factors is a change in the cellular structure of smooth muscle cells, causing the cells to shift from quiescent to migratory. This cellular change is of particular importance in vascular medicine, since activation of quiescent smooth muscle cells in arteries can lead to uncontrolled proliferation, leading to the blockage or narrowing of arteries known as stenosis or restenosis.

The standard of care for the non-surgical treatment of blocked arteries is to re-open the blockage with an angioplasty balloon, often followed by the placement of a wire metal structure called a stent to retain the opening in the artery. An unfortunate consequence of this procedure is the nearly total destruction of the endothelial layer by expansion of the angioplasty balloon and precipitation of foreign body inflammatory response to the stent. Therefore, after removal of the balloon catheter used in the angioplasty, the artery is rapidly exposed to an influx of activating factors. Since mechanical intervention has destroyed the natural blood/artery barrier, all too often the result is a local uncontrolled proliferative response by smooth muscle cells leading to restenosis.

Other types of wounds undergo similar processes. In general, wounds can be divided into two types: acute and chronic. In cases where a wound is not initially surgically closed (delayed primary closure), the wound is left open for a time sufficient to allow the inflammatory process and angiogenesis to begin before surgical closure. Wounds healing by secondary intention are usually not amenable to surgical closure. As a result, the wound is left to granulate and epithelialize from the wound bed and edges. Numerous dressing products were developed during the past few years to accelerate this type of healing process.

For these types of acute wounds, occlusive dressings increase re-epithelialization rates by 30% to 50% and collagen synthesis by 20% to 60% compared to wounds exposed to air by providing an optimal healing environment that exposes the wound continuously to the surrounding fluid of proteinases, chemotactic factors, complements, and growth factors. An electrical gradient that may stimulate fibroblast and epithelial cell migration is maintained. The use of non-adherent dressing prevents the stripping of the newly formed epithelial layer.

An occlusive dressing is generally divided into a hydrating layer (antibiotic ointments or petrolatum jelly), a nonadherent contact layer, an absorbent and cushioning layer (gauze), and a securing layer (tape or wrap). Occlusive dressings are commonly applied within 2 hours of wounding and left on for at least 24 hours, rarely as long as 48 hours, for optimal healing of acute wounds. Initial wound hypoxia is important for fibroblast proliferation and angiogenesis; however, continued hypoxia at the wound site delays wound healing. As a result, if an occlusive dressing is applied to an ischemic wound, healing is severely impaired.

Chronic wounds are defined as wounds that fail to heal after 3 months. Venous stasis ulcers, diabetic ulcers, pressure ulcers, and ischemic ulcers are the most common chronic wounds. Many of the dressing options that attempt to heal venous stasis ulcers are a variation on the classic paste compression bandage, Unna's boot. These wounds can sometimes have large amounts of exudates that require frequent debridement. Alginates, foams, and other absorptives can be used in this situation. Because chronic wounds heal by slightly different mechanisms than those of acute wounds, experimentation with growth factors is being investigated. Regranex® and Procuren® (Curative Health Services, Inc., Hauppauge, N.Y.) are the only medications approved by the US Food and Drug Administration (FDA).

Thus, a need exists in the art for new and better methods and devices for restoring the natural process of wound healing in damaged arteries and other blood vessels as well as in healing of other types of acute and chronic wounds.

### SUMMARY OF THE INVENTION

In one embodiment, the invention provides wound healing compositions containing a biodegradable, biocompatible polymer and at least one wound healing agent dispersed in the polymer. The biodegradable polymer is a PEA having a structural formula described by structural formula (I), 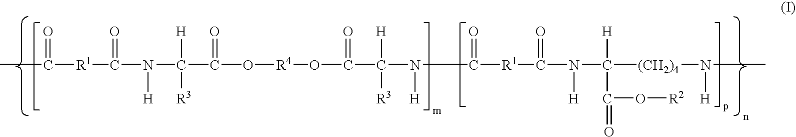
and wherein n ranges from about 5 to about 150, m ranges about 0.1 to about 0.9: p ranges from about 0.9 to about 0.1; wherein R¹ is selected from the group consisting of (C₂-C₂₀) alkylene or (C₂-C₂₀) alkenylene; R² is hydrogen or (C₆-C₁₀)aryl (C₁-C₆) alkyl, or a protecting group; R³ is selected from the group consisting of hydrogen, (C₁-C₆) alkyl, (C₂-C₆) alkenyl, (C₂-C₆) alkynyl and (C₆-C₁₀)aryl(C₁-C₆) alkyl; and R⁴ is selected from the group consisting of (C₂-C₂₀) alkylene, (C₂-C₂₀) alkenylene or alkyloxy, and bicyclic-fragments of 1,4:3,6-dianhydrohexitols of general formula (II): 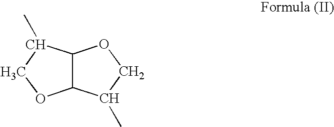
except that for unsaturated polymers having the chemical structure of structural formula (I), R¹ and R⁴ are selected from (C₂-C₂₀) alkylene and (C₂-C₂₀) alkenylene; wherein at least one of R¹ and R⁴ is (C₂-C₂₀) alkenylene; n is about 5 to about 150; each R² is independently hydrogen, or (C₆-C₁₀)aryl(C₁-C₆)alkyl; and each R³ is independently hydrogen, (C₁-C₆)alkyl, (C₂-C₆)alkenyl, (C₂-C₆)alkynyl, or (C₆-C₁₀)aryl(C₁-C₆)alkyl;
or a PEUR having a chemical formula described by general structural formula (III), 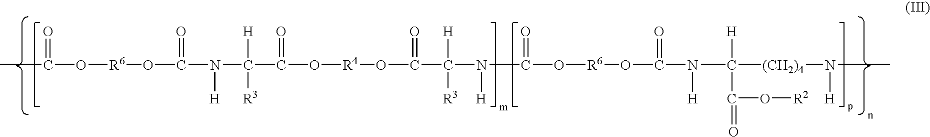
   and wherein n ranges from about 5 to about 150, m ranges about 0.1 to about 0.9: p ranges from about 0.9 to about 0.1; wherein R² is hydrogen or (C₆-C₁₀)aryl(C₁-C₆) alkyl, or a protecting group; R³ is selected from the group consisting of hydrogen, (C₁-C₆) alkyl, (C₂-C₆) alkenyl, (C₂-C₆) alkynyl and (C₆-C₁₀) aryl(C₁-C₆) alkyl; R⁴ is selected from the group consisting of (C₂-C₂₀) alkylene, (C₂-C₂₀) alkenylene or alkyloxy, and bicyclic-fragments of 1,4:3,6-dianhydrohexitols of general formula (II); and R⁶ is independently selected from (C₂-C₂₀) alkylene, (C₂-C₂₀) alkenylene or alkyloxy, and bicyclic-fragments of 1,4:3,6-dianhydrohexitols of general formula (II).

In another embodiment, the invention provides methods for promoting natural healing of a wound by contacting the wound with an invention wound healing composition under conditions suitable for promoting natural healing of the wound.

In still another embodiment, the invention provides a multilayer bioactive wound dressing that includes a non-stick layer comprising a biodegradable hydrogel; a supporting layer of a biodegradable polymer having a chemical structure described by formula (I) or (III) overlying the non-stick layer; and at least one wound healing agent that produce a wound healing effect in situ dispersed within the polymer, the hydrogel, or both.

### A BRIEF DESCRIPTION OF THE FIGURES

FIG. 1 is a schematic cross-section of an invention multilayered polymer-coated stent.

FIG. 2 is a graph illustrating the effect of various bioagents used in invention stents (see Table 1) on adhesion and proliferation of endothelial cells (ECs) growing on gelatin coated surfaces. Control=zero concentration of bioactive agent.

FIG. 3 is a graph illustrating the effect of various bioagents used in invention stents (see Table 1) on adhesion and proliferation of smooth muscle cells (SMCs) growing on gelatin coated surfaces. Control=zero concentration of bioactive agent.

FIG. 4 is a flow chart of the protocol for adhesion assays conducted with ECs and SMCs.

FIG. 5 is a graph summarizing the results of a representative adhesion assay quantitation based on ATP standard curve. At each time point of the adhesion assay, an ATP assay was done to determine the number of adherent cells.

FIG. 6 shows the chemical structure of dansyl, an acronym for 5 dimethylamino-1 naphthalenesulfonyl, a reactive fluorescent dye, linked to PEA.

FIGS. 7A and B are flowcharts summarizing surface chemistry optimization protocols. FIG. 7A shows a flowchart of the surface chemistry for conjugation of peptides to the acid version of the polymers (PEA-H). FIG. B shows a flowchart of the protocol for surface conjugation of peptides to mixtures of PEA polymers.

### DETAILED DESCRIPTION OF THE INVENTION

The present invention is based on the discovery that biodegradable polymers, hydrogels, or both, can be used to create compositions suitable for use in wound dressings, implants, and surgical device coatings that promote endogenous healing processes at a wound site. The polymers biodegrade over time, releasing wound healing agents that establish or re-establish the natural healing process in a wound, such as a chronic wound. A released wound healing agent can either be absorbed into a target cell in a wound site where it acts intracellularly, either within the cymosely, the nucleus, or both, or the wound healing agent can bind to a cell surface receptor molecule to elicit a cellular response without entering the cell. Alternatively, the wound healing agent dispersed in the polymer or hydrogel matrix promotes endogenous healing processes at the wound site by contact with the surroundings into which the wound dressing, implant or surgical device is placed. Depending upon the rate of biodegradation of the polymer, the hydrogel matrix or coating, the healing properties of the invention wound healing compositions can take place even before biodegradation of the polymer or hydrogel.

This invention describes wound healing compositions that can be fashioned into wound dressings, implants and surgical device coatings, which wound healing compositions comprise (a) a biodegradable, biocompatible polymer, a hydrogel, or both, as a carrier into which is dispersed, mixed, dissolved, homogenized, or covalently bound (“dispersed”) (b) at least one wound healing agent. Optionally, additional bioactive agents can be dispersed within the polymer, hydrogel, or both.

The term “wound healing agent,” as used herein, means bioactive agents that are effective for promoting natural wound healing processes over days, weeks, or months. The invention wound healing compositions can be prepared in the form of drug delivery wound dressings, implants, and coatings that cover at least a portions of a surgical device and can be in any appropriate form into which the polymer or hydrogel, or both, including the wound healing agents as well as optional additional bioactive agents, can be formed with polymer and hydrogel technological processing methods as known in the art and as described herein.

In one embodiment, the invention wound healing composition is used to fashion a polymer implant designed for implantation into an internal body site wherein the polymer implant comprises a biodegradable, biocompatible polymer as described herein from which a dispersed wound healing agent is released over a considerable period of time, for example, over a period of three months to about twelve months. The wound healing agent is released in situ as a result of biodegradation of the polymer carrier. A cross-linked poly (ester amide), polycaprolactone, or poly (ester urethane) as described herein can be used for this purpose so that the polymer implant is completely biodegradable. PEA and PEUR polymers described by formulas (I) and (III) containing a plurality of unsaturated moieties are particularly useful for creating such cross-linked polymers. In this case, over time, the polymer implant will be re-absorbed by the body through natural enzymatic action, allowing the re-established endothelial cell layer to resume its natural function.

In another embodiment, the invention wound healing composition is used in a wound dressing comprising the above-described biodegradable, biocompatible polymer as a carrier with at least one wound healing agent dispersed in the polymer. Alternatively the wound dressing can also comprise a biodegradable hydrogel, such as is described herein, as the carrier with at least one wound healing agent dispersed in the hydrogel. Alternatively still, the invention wound dressing can comprise separate portions, for example separate layers, of the biodegradable biocompatible polymer and the hydrogel with the wound healing agent dispersed in the polymer portion, or in both. Alternatively still, two different wound healing agents as described herein may be dispersed in the separate portions of the wound dressing. Optionally, additional bioactive agents, as described herein, can be dispersed in the polymer portion, the hydrogel portion, or in both.

In another embodiment, the invention provides bioactive implantable stents including a stent structure with a surface coating of a biodegradable, bioactive polymer, wherein the polymer includes at least one bioactive agent dispersed in the polymer, and wherein at least one therapeutic bioactive agent is produced in situ as a result of biodegradation of the polymer.

The invention provides stents and methods of their use that are designed to re-establish a blood/artery barrier concurrently with the placement of the stent in a damaged artery. The invention stents comprise a biodegradable, biocompatible polymeric sheath or coating that encapsulates the stent structure. In a preferred embodiment of the invention methods, the stent is emplaced at the conclusion of the angioplasty procedure, or other medical procedure that damages the arterial endothelium, without allowing a lapse of time sufficient for infiltration of inflammatory factors from the blood stream into the artery wall. In this method, the stent is placed at the location of the damage and preferably immediately covers and protects the area of damaged endothelium so as to prevent infiltration of inflammatory factors from the blood stream into the artery wall, thereby limiting the proliferation of smooth muscle cells and consequent restenosis.

In other words, the invention stents perform as an artificial endothelial layer while promoting the natural cycle of endothelial healing as described herein. The polymeric sheath may have additional features that contribute to the healing of the artery. In one embodiment, the invention sheath or covering comprises multiple layers, each of which can perform a distinct function in re-establishing a stable lesion and contributing to healing of the injured artery wall.

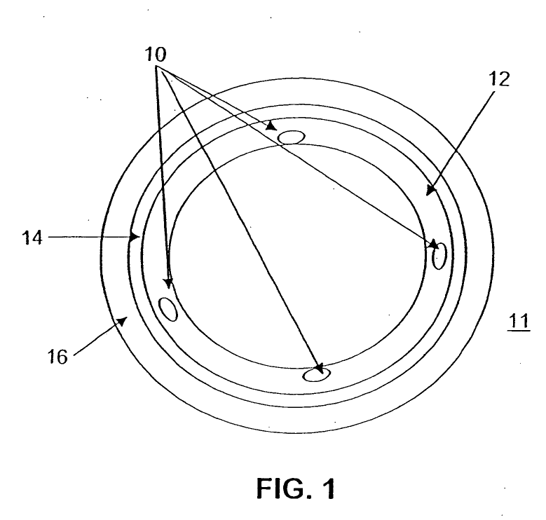

FIG. 1 shows a schematic cross-section of an example of an invention stent 11 with stent struts 10 and a multilayered sheath or covering. When the multilayered stent is implanted, the outer layer 16 of the stent sheath lies directly next to the artery wall. A diffusion barrier layer 14 lies between and is in contact with outer layer 16 and inner layer 12.

The outer layer comprises a polymer layer loaded with a bioactive agent and/or an additional bioactive agent, or combination thereof, specifically including those that limit cellular proliferation or reduce inflammation as disclosed herein. These cellular proliferation limiting and/or inflammation reducing drugs and bioactive agents can be solubilized in the polymer solid phase and, hence are preferably not bound to the polymer of the outer layer, but are loaded into the polymer and sequestered there (dispersed therein) until the stent is put into place. Once implanted, the active agents in the outer layer 16 diffuse into the artery wall.

Preferred additional bioactive agents for incorporation into the outer layer of invention multilayered stents include anti-proliferants, rapamycin and any of its analogs or derivatives, paclitaxel or any of its taxene analogs or derivatives, everolimus, Sirolimus, tacrolimus, or any of its—limus named family of drugs, and statins such as simvastatin, atorvastatin, fluvastatin, pravastatin, lovastatin, rosuvastatin, geldanamycins, such as 17AAG (17-allylamino-17-demethoxygeldanamycin); Epothilone D and other epothilones, 17-dimethylaminoethylamino-17-demethoxy-geldanamycin and other polyketide inhibitors of heat shock protein 90 (Hsp90), Cilostazol, and the like. In the outer layer of the multilayered stent, non-covalently bound bioactive agents and/or additional bioactive agents can be dispersed (e.g., intermingled with or “loaded into”) any biocompatible biodegradable polymer as is known in the art since the outer layer in this embodiment of the invention comes into contact with blood primarily only at the edges of the stent.

Lying along and covering the interior surface of the outer layer of the covering is a diffusion barrier layer 12 of biodegradable polymer that acts as a diffusion barrier to the drug or biologic contained in the outer layer. The purpose of this diffusion barrier is to direct elution of the bioactive agents in the inner layer into the artery wall to prevent proliferation of smooth muscle cells, while limiting or preventing passage of the drug/biologic into the inner layer. The diffusion barrier layer 12 can accomplish its purpose of partitioning of the drug through hydrophobic/hydrophilic interaction related to the solubility of the bioactive agent in the polymer solid phase. For example, if the bioactive agent or additional bioactive agent in the outer layer is hydrophobic, the polymer barrier layer is selected to be less hydrophobic than the agent(s), and if the bioactive agent or additional bioactive agent in the outer layer is hydrophilic, the barrier layer is selected to be hydrophobic. For example, the barrier layer can be selected from such polymers as polyester, poly (amino acid), poly (ester amide), poly (ester urethane), polyurethane, polylactone, poly (ester ether), or copolymers thereof.

For fabrication of the inner layer 12 of the invention multilayered stent, which is exposed to the circulating blood with its endothelial progenitor cells, a polymer of the type specifically described herein as having a chemical structure described by formula I or III is used. One or more wound healing agent involved in the natural processes of endothelialization is dispersed in the polymer in the inner layer using techniques described herein. To accomplish this end, the bioactive agent for use in the inner layer of the multilayered stent is selected to activate and attract circulating endothelial progenitor cells to the inner layer of the sheath or coating on the porous stent structure, thereby beginning the process of re-establishing the natural endothelial cell layer.

In one embodiment, the stent structure used in manufacture of the invention multilayered stent is made of a biodegradable material with sufficient strength and stiffness to replace a conventional stent, such as a stainless steel or wire mesh stent structure. A cross-linked poly (ester amide), polycaprolactone, or poly (ester urethane) as described herein can be used for this purpose so that the stent is completely biodegradable and biocompatible. In this case, over time, each of the layers, and the stent structure as well, will be re-absorbed by the body through natural enzymatic action, allowing the re-established endothelial cell layer to resume its dual function of acting as a blood/artery barrier and providing natural control and stabilization of the intra-cellular matrix within the artery wall through the production of nitric oxide.

As used herein, “biodegradable” means that the polymer or hydrogel, whether in the form of a coating on a surgical device, such as a stent, in the form of a wound dressing, or a polymer implant, is capable of being broken down into innocuous biocompatible products in the normal functioning of the body. In one embodiment, the entire coated device is biodegradable. The biodegradable polymers have hydrolyzable ester linkages, which provide the biodegradability, and are typically chain terminated with carboxyl groups.

As used herein “dispersed” means a bioactive agent, i.e., a wound healing agent, or mixture of wound healing agent and additional bioactive agents, is dispersed, mixed, dissolved, homogenized, or (“dispersed”) within a polymer or hydrogel, or both, as described herein, or covalently bonded to the biodegradable polymer, as described herein.

Polymers suitable for use in the practice of the invention bear functionalities that allow for facile covalent attachment of bioactive agents to the polymer. For example, a polymer bearing carboxyl groups can readily react with a bioactive agent having an amino moiety, thereby covalently bonding the bioactive agent to the polymer via the resulting amide group. As will be described herein, the biodegradable polymer and the bioactive agent can contain numerous complementary functional groups that can be used to covalently attach the bioactive agent to the biodegradable polymer.

As used herein, “bioactive” means the agent plays an active role in the endogenous healing processes at a wound site by releasing a drug or bioactive agent during biodegradation of the polymer, hydrogel, or both, contained therein. Wound healing agents contemplated for dispersion within the polymers, hydrogels, or both, when freed or eluted from the polymer or hydrogel during its degradation, enhance endogenous production of a therapeutic natural wound healing agent, such as nitric oxide, which is endogenously produced by endothelial cells. Alternatively the bioactive agent(s) released from the compositions during degradation may be directly active in promoting natural wound healing processes by endothelial cells. These wound healing agents can be any bioactive agent that donates, transfers, or releases nitric oxide, elevates endogenous levels of nitric oxide, stimulates endogenous synthesis of nitric oxide, or serves as a substrate for nitric oxide synthase or that inhibits proliferation of smooth muscle cells.

Such wound-healing agents include, for example, aminoxyls, furoxans, nitrosothiols, nitrates and anthocyanins; nucleosides, such as adenosine; and nucleotides, such as adenosine diphosphate (ADP) and adenosine triphosphate (ATP); neurotransmitter/neuromodulators, such as acetylcholine and 5-hydroxytryptamine (serotonin/5-HT); histamine and catecholamines, such as adrenalin and noradrenalin; lipid molecules, such as sphingosine-1-phosphate and lysophosphatidic acid; amino acids, such as arginine and lysine; peptides such as the bradykinins, substance P and calcium gene-related peptide (CGRP), and proteins, such as insulin, vascular endothelial growth factor (VEGF), and thrombin. The term “nitric oxide-releasing compound” means any compound (e.g., polymer) to which is bound a nitric oxide releasing functional group. Suitable nitric oxide-releasing compounds are S-nitrosothiol derivative (adduct) of bovine or human serum albumin and as disclosed, e.g., in U.S. Pat. No. 5,650,447. See, e.g., “Inhibition of neointimal proliferation in rabbits after vascular injury by a single treatment with a protein adduct of nitric oxide”; David Marks et al. J. Clin. Invest. (1995) 96:2630-2638.

In addition, examples of wound healing agents for the capture of PECs are monoclonal antibodies directed against a known PEC surface marker. Complementary determinants (CDs) that have been reported to decorate the surface of endothelial cells include CD31, CD34+, CD34−, CD102, CD105, CD106, CD109, CDw130, CD141, CD142, CD143, CD 144, CDw145, CD 146, CD147, and CD166. These cell surface markers can be of varying specificity and the degree of specificity for a particular cell/developmental type/stage is in many cases not fully characterized. In addition these cell marker molecules against which antibodies have been raised will overlap (in terms of antibody recognition) especially with CDs on cells of the same lineage: monocytes in the case of endothelial cells. Circulating endothelial progenitor cells are some way along the developmental pathway from (bone marrow) monocytes to mature endothelial cells. CDs 106, 142 and 144 have been reported to mark mature endothelial cells with some specificity. CD34 is presently known to be specific for progenitor endothelial cells and therefore is currently preferred for capturing progenitor endothelial cells out of circulating blood in the site into which the wound healing composition is implanted. Examples of such antibodies include single-chain antibodies, chimeric antibodies, monoclonal antibodies, polyclonal antibodies, antibody fragments, Fab fragments, IgA, IgG, IgM, IgD, IgE and humanized antibodies, as are known in the art.

Small proteinaceous motifs, such as the B domain of bacterial Protein A and the functionally equivalent region of Protein G, that are known to bind to, and thereby capture, such antibody molecules can be covalently attached to polymers and will act as ligands to capture antibodies by the Fc region out of the patient's blood stream. Therefore, the antibody types that can be attached to polymers and polymer coatings using a Protein A or Protein G functional region are those that contain an Fc region. The captured antibodies will in turn bind to and hold captured progenitor endothelial cells near the polymer surface while other activating factors, such as the bradykinins, activate the progenitor endothelial cells.

However, for embodiments of the invention wound healing composition formulated as wound dressings and polymer implants, it should be noted that access of the wound healing composition to circulating blood will be minimal, especially in treatment of chronic wounds. Therefore, the following drugs and bioactive agents will be particularly effective for dispersion within the polymers, hydrogels, or both, used in making invention wound dressings, whether dispersed within a time release biodegradable hydrogel, as described herein, or a biodegradable, compatible polymer having a chemical structure described by structures I and III herein.

For wound healing, the bioactive agents that are incorporated into the invention compositions in wound dressings and device coatings are not limited to, but include, various classes of compounds that contribute to wound healing when presented in a time-release fashion to the wound surface. Such wound healing agents include wound healing cells, which are protected, nurtured and delivered by the biodegradable polymer(s), hydrogels, or both, in the invention wound dressings. Wound healing cells that can be used in practice of the invention include, for example, pericytes and endothelial cells, including progenitor endothelial cells.

An additional category of wound healing cells are inflammatory healing cells. To recruit such cells to the wound bed, the composition can include ligands for such cells, such as antibodies and smaller molecule ligands, whether biologics or synthetic, that specifically bind to such “cellular adhesion molecules” (CAMs). Exemplary ligands for wound healing cells include those that specifically bind to Intercellular adhesion molecules (ICAMs), such as ICAM-1 (CD54 antigen); ICAM-2 (CD 102 antigen); ICAM-3 (CD50 antigen); ICAM-4 (CD242 antigen); and ICAM-5; Vascular cell adhesion molecules (VCAMs), such as VCAM-1 (CD106 antigen)]; Neural cell adhesion molecules (NCAMs), such as NCAM-1 (CD56 antigen); or NCAM-2; Platelet endothelial cell adhesion molecules PECAMs, such as PECAM-1 (CD31 antigen); Leukocyte-endothelial cell adhesion molecules (ELAMs), such as LECAM-1; or LECAM-2 (CD62E antigen), and the like.].

For example, the wound healing cells can be dispersed within a hydrogel loaded with a suitable growth medium for the cells. Synthetic tissue grafts, such as Apligraf® (Novartis), which is specifically formulated for healing of diabetic chronic wounds, can be supported by attachment to polymer layers in invention wound dressings.

In another aspect, the wound healing agents include extra cellular matrix proteins, which are macromolecules that can be dispersed in the polymers, hydrogels, or both, in the invention wound healing compositions. Examples of useful extra-cellular matrix proteins for this purpose include, for example, glycosaminoglycans, usually linked to proteins (proteoglycans), and fibrous proteins (e.g., collagen; elastin; fibronectins and laminin). Bio-mimics of extra-cellular proteins can also be used. These are usually non-human but biocompatible glycoproteins, such as derivatives of alginates and chitin. Wound healing peptides that are specific fragments of such extra-cellular matrix proteins or their bio-mimics can also be used.

Proteinaceous growth factors are an additional category of wound healing agents suitable for incorporation into the various invention wound healing compositions used in wound dressings, implants and surgical device coatings described herein. For example, Platelet Derived Growth Factor-BB (PDGF-BB), Tumor Necrosis Factor-alpha (TNF-alpha), Epidermal Growth Factor (EGF), Keratinocyte Growth Factor (KGF), Thymosin B4; and, various angiogenic factors such as vascular Endothelial Growth Factors (VEGFs), Fibroblast Growth Factors (FGFs), Tumor Necrosis Factor-beta (TNF-beta), and Insulin-like Growth Factor-1 (IGF-1). Many of these proteinaceous growth factors are available commercially or can be produced recombinantly using techniques well known in the art. Alternatively, expression systems comprising vectors, particularly adenovirus vectors, incorporating genes encoding such proteinaceous growth factors can be dispersed into the invention wound healing compositions for administration of the growth factors to the wound bed.

Drugs that enable healing are an additional category of wound healing agents suitable for dispersion into the various invention wound healing compositions used in wound dressings, implants and device coatings described herein. Such healing enabler drugs include, for example, antimicrobials and anti-inflammatory agents as well as certain healing promoters, such as, for example, vitamin A and synthetic inhibitors of lipid peroxidation.

A variety of antibiotics can also be dispersed in the invention wound healing compositions to indirectly promote natural healing processes by preventing or controlling infection. Suitable antibiotics include many classes, such as aminoglycoside antibiotics or quinolones or beta-lactams, such as cefalosporines, e.g., ciprofloxacin, gentamycin, tobramycin, erythromycin, vancomycin, oxacillin, cloxacillin, methicillin, lincomycin, ampicillin, and colistin. Suitable antibiotics have been described in the literature

Suitable antimicrobials include, for example, Adriamycin PFS/RDF® (Pharmacia and Upjohn), Blenoxane® (Bristol-Myers Squibb Oncology/Immunology), Cerubidine® (Bedford), Cosmegen® (Merck), DaunoXome® (NeXstar), Doxil® (Sequus), Doxorubicin Hydrochloride® (Astra), Idamycin® PFS (Pharmacia and Upjohn), Mithracin® (Bayer), Mitamycin® (Bristol-Myers Squibb Oncology/Immunology), Nipen® (SuperGen), Novantrone® (Immunex) and Rubex® (Bristol-Myers Squibb Oncology/Immunology).

In one embodiment, the peptide can be a glycopeptide. “Glycopeptide” refers to oligopeptide (e.g. heptapeptide) antibiotics, characterized by a multi-ring peptide core optionally substituted with saccharide groups, such as vancomycin.

Examples of glycopeptides included in this category of antimicrobials may be found in “Glycopeptides Classification, Occurrence, and Discovery,” by Raymond C. Rao and Louise W. Crandall, (“Bioactive agents and the Pharmaceutical Sciences” Volume 63, edited by Ramakrishnan Nagaraj an, published by Marcal Dekker, Inc.). Additional examples of glycopeptides are disclosed in U.S. Pat. Nos. 4,639,433; 4,643,987; 4,497,802; 4,698,327, 5,591,714; 5,840,684; and 5,843,889; in EP 0 802 199; EP 0 801 075; EP 0 667 353; WO 97/28812; WO 97/38702; WO 98/52589; WO 98/52592; and in J. Amer. Chem. Soc., 1996, 118, 13107-13108; J. Amer. Chem. Soc., 1997, 119, 12041-12047; and J. Amer. Chem. Soc, 1994, 116, 4573-4590. Representative glycopeptides include those identified as A477, A35512, A40926, A41030, A42867, A47934, A80407, A82846, A83850, A84575, AB-65, Actaplanin, Actinoidin, Ardacin, Avoparcin, Azureomycin, Balhimyein, Chloroorientiein, Chloropolysporin, Decaplanin, -demethylvancomycin, Eremomycin, Galacardin, Helvecardin, Izupeptin, Kibdelin, LL-AM374, Mannopeptin, MM45289, MM47756, MM47761, MM49721, MM47766, MM55260, MM55266, MM55270, MM56597, MM56598, OA-7653, Orenticin, Parvodicin, Ristocetin, Ristomycin, Synmonicin, Teicoplanin, UK-68597, UD-69542, UK-72051, Vancomycin, and the like. The term “glycopeptide” or “glycopeptide antibiotic” as used herein is also intended to include the general class of glycopeptides disclosed above on which the sugar moiety is absent, i.e. the aglycone series of glycopeptides. For example, removal of the disaccharide moiety appended to the phenol on vancomycin by mild hydrolysis gives vancomycin aglycone. Also included within the scope of the term “glycopeptide antibiotics” are synthetic derivatives of the general class of glycopeptides disclosed above, included alkylated and acylated derivatives. Additionally, within the scope of this term are glycopeptides that have been further appended with additional saccharide residues, especially aminoglycosides, in a manner similar to vancosamine.

The term “lipidated glycopeptide” as used herein, refers specifically to those glycopeptide antibiotics which have been synthetically modified to contain a lipid substituent. As used herein, the term “lipid substituent” refers to any substituent that contains 5 or more carbon atoms, preferably, 10 to 40 carbon atoms. The lipid substituent may optionally contain from 1 to 6 heteroatoms selected from halo, oxygen, nitrogen, sulfur, and phosphorous. Lipidated glycopeptide antibiotics are well-known in the art. See, for example, in U.S. Pat. Nos. 5,840,684, 5,843,889, 5,916,873, 5,919,756, 5,952,310, 5,977,062, 5,977,063, EP 667, 353, WO 98/52589, WO 99/56760, WO 00/04044, and WO 00/39156.

Anti-inflammatory agents useful for dispersion in polymers and hydrogels used in invention wound healing compositions, depending on the body site to be treated, include, e.g. analgesics (e.g., NSAIDS and salicyclates), antirheumatic agents, gastrointestinal agents, gout preparations, hormones (glucocorticoids), nasal preparations, ophthalmic preparations, otic preparations (e.g., antibiotic and steroid combinations), respiratory agents, and skin & mucous membrane agents. See, Physician's Desk Reference, 2005 Edition. Specifically, the anti-inflammatory agent can include dexamethasone, which is chemically designated as (11θ, 16I)-9-fluro-11,17,21-trihydroxy-16-methylpregna-1,4-diene-3,20-dione. Alternatively, the anti-inflammatory agent can include sirolimus (rapamycin), which is a triene macrolide antibiotic isolated from Steptomyces hygroscopicus.

In certain embodiments of the invention, the bioactive agents are covalently bonded to the polymers used in the invention wound dressings, implants and device coatings. The following examples illustrate the ease with which certain categories of bioactive agents can be incorporated into the invention polymers. Aminoxyls contemplated for use as bioactive agents have the structure:

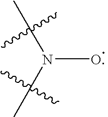

Exemplary aminoxyls include the following compounds: 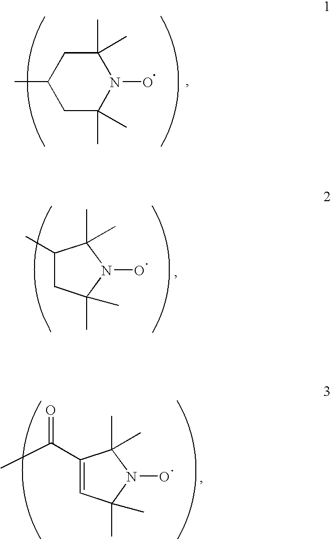
2,2,6,6-tetramethylpiperidine-1-oxy (1); 2,2,5,5-tetramethylpyrrolidine-1-oxy (2); and 2,2,5,5-tetramethylpyrroline-1-oxy-3-carbonyl (3). Further aminoxyls contemplated for use include 4-amino-2,2,6,6-tetramethylpiperidine-1-oxy (TEMPAMINE); 4-(N,N-dimethyl-N-hexadecyl)ammonium-2,2,6,6-tetramethylpiperidine-1-oxy, iodide (CAT16); 4-(N,N-dimethyl-N-(2-hydroxyethyl))ammonium-2,2,6,6-tetramethylpiperidine-1-oxy(TEMPO choline); 4-(N,N-dimethyl-N-(3-sulfopropyl)ammonium-2,2,6,6-tetramethylpiperidine-1-oxy; N-(4-(iodoacetyl)amino-2,2,6,6-tetramethylpiperidine-1-oxy (TEMPO 1A); N-(2,2,6,6-tetramethylpiperidine-1-oxy-4-yl)maleimide (TEMPO maleimide, MAL-6); and 4-trimethylammonium-2,2,6,6-tetramethylpiperidine-1-oxy, iodide (CAT 1); 3-amino-2,2,5,5-tetramethylpyrrolidine-1-oxy; and N-(3-(iodoacetyl)amino)-2,2,5,5-tetramethylpyrrolidine-1-oxy(PROXYL 1A); succinimidyl 2,2,5,5-tetramethyl-3-pyrroline-1-oxy-3-carboxylate and 2,2,5,5-tetramethyl-3-pyrroline-1-oxy-3-carboxylic acid, and the like.

Furoxans contemplated for use as bioactive agents have the structure:

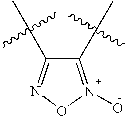

An exemplary furoxan is 4-phenyl-3-furoxancarbonitrile, as set forth below:

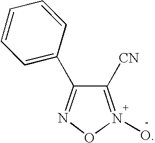

Nitrosothiols include compounds bearing the —S—N═O moiety, such as the exemplary nitrosothiol set forth below:

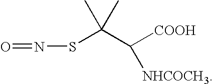

Anthocyanins are also contemplated for use as bioactive agents. Anthocyanins are glycosylated anthocyanidins and have the structure: 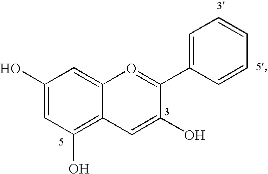
wherein the sugars are attached to the 3-hydroxy position. Anthocyanins are known to stimulate NO production in vivo and therefore are suitable for use as wound healing agents in the practice of the invention.

In further embodiments, the wound healing agent is a ligand for attaching to or capturing progenitor endothelial cells floating within the blood stream within a blood vessel. In one embodiment, the ligand is a “sticky” peptide or polypeptide, such as Protein A and Protein G. Protein A is a constituent of staphylococcus A bacteria that binds the Fc region of particular antibody or immunoglobulin molecules, and is used extensively to identify and isolate these molecules. For example the Protein A ligand can be or contain the amino acid sequence:

(SEQ ID NO: 1)

MTPAVTTYKLVINGKTLKGETTTKAVDAETAEKAFKQYANDNGVDGVWTY
DDATKTFTVTE

or a functionally equivalent peptidic derivative thereof, such as, by way of an example, the functionally equivalent peptide having the amino acid sequence:

(SEQ ID NO: 2)

TYKLILNGKTLKGETTTEAVDAATAEKVFKQYANDNGVDGEWTYDDATKT
FTVTE

Protein G is a constituent of group G streptococci bacteria, and displays similar activity to Protein A, namely binding the Fc region of particular antibody or immunoglobulin molecules. For example, the Protein G ligand can be, or contain Protein G having an amino acid sequence:

(SEQ ID NO: 3)

MTPAVTTYKLVINGKTLKGETTTKAVDAETAEKAFKQYANDNGVDGVWTY
DDATKTFTVTE

or a functionally equivalent peptidic derivative thereof, such as, by way of an example, the functionally equivalent peptide having the amino acid sequence:

(SEQ ID NO: 4)

TYKLILNGKTLKGETTTEAVDAATAEKVFKQYANDNGVDGEWTYDDATKT
FTVTE

Other wound healing peptides contemplated for dispersion as wound healing agents in the polymers and hydrogels of the invention compositions used in fabrication of wound dressings, implants, and surgical device coatings include the bradykinins. Bradykinins are vasoactive nonapeptides formed by the action of proteases on kininogens, to produce the decapeptide kallidin (KRPPGFSPFR) (SEQ ID NO: 5), which can undergo further C-terminal proteolytic cleavage to yield the bradykinin 1 nonapeptide: (KRPPGFSPF) (SEQ ID NO: 6), or N-terminal proteolytic cleavage to yield the bradykinin 2 nonapeptide: (RPPGFSPFR) (SEQ ID NO: 7). Bradykinins 1 and 2 are functionally distinct as agonists of specific bradykinin cell surface receptors B1 and B2 respectively: both kallidin and bradykinin 2 are natural ligands for the B2 receptor whereas their C-terminal metabolites (bradykinin 1 and the octapeptide RPPGFSPF (SEQ ID NO:8) respectively) are ligands for the B1 receptor. A portion of circulating bradykinin peptides can be subject to a further post-translational modification: hydroxylation of the second proline residue in the sequence (Pro3 to Hyp3 in the bradykinin 2 amino acid numbering). Bradykinins are very potent vasodilators, increasing permeability of post-capillary venules, and acting on endothelial cells to activate calmodulin and thereby nitric oxide synthase.

Bradykinin peptides are incorporated into the polymers used in the invention wound healing compositions by attachment at one end of the peptide. The unattached end of the bradykinin extends freely from the polymer to contact endothelial cells. For example, when the bradykinin is dispersed in an invention wound healing composition used to coat a stent, the bradykinin peptide contacts endothelial cells in the vessel wall, as well as progenitor endothelial cells floating in the blood vessel into which the stent is implanted to activate the endothelial cells with which contact is made. Endothelial cells activated in this way activate further progenitor endothelial cells with which they come into contact, thereby causing a cascade of endothelial cell activation at the site of the injury that results in endogenous production of nitric oxide.

In a still further aspect, the wound healing agent can be a nucleoside, such as adenosine, which is also known to be a potent activator of endothelial cells to produce nitric oxide endogenously.

Biodegradable polymers contemplated for use in the invention wound healing compositions include polyesters, poly(amino acids), polyester amides, polyurethanes, or copolymers thereof. In particular, examples of biodegradable polyesters include poly(α-hydroxy C₁-C₅ alkyl carboxylic acids), e.g., polyglycolic acids, poly-L-lactides, and poly-D,L-lactides; poly-3-hydroxy butyrate; polyhydroxyvalerate; polycaprolactones, e.g., poly(ε-caprolactone); and modified poly(α-hydroxyacid)homopolymers, e.g., homopolymers of the cyclic diester monomer, 3-(S)[alkyloxycarbonyl)methyl]-1,4-dioxane-2,5-dione which has the formula 4 where R is lower alkyl, depicted in Kimura, Y., “Biocompatible Polymers” in Biomedical Applications of Polymeric Materials, Tsuruta, T., et al, eds., CRC Press, 1993 at page 179.

In one embodiment, the invention provides polymer wound healing compositions containing a biodegradable, biocompatible polymer and a wound healing agent dispersed in the polymer, wherein the biodegradable polymer is a PEA having a chemical formula described by structural formula (I), 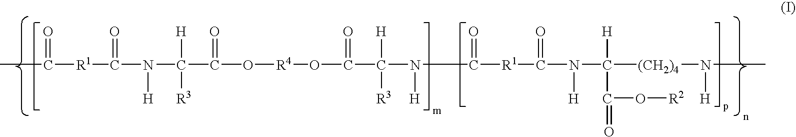
and wherein n ranges from about 5 to about 150, m ranges about 0.1 to about 0.9: p ranges from about 0.9 to about 0.1; wherein R¹ is selected from the group consisting of (C₂-C₂₀) alkylene or (C₂-C₂₀) alkenylene; R² is hydrogen or (C₆-C₁₀)aryl (C₁-C₆) alkyl, or a protecting group, such as t-butyl. Additional protecting groups are well known in the art. R³ is selected from the group consisting of hydrogen, (C₁-C₆) alkyl, (C₂-C₆) alkenyl, (C₂-C₆) alkynyl and (C₆-C₁₀)aryl(C₁-C₆) alkyl; and R⁴ is selected from the group consisting of (C₂-C₂₀) alkylene, (C₂-C₂₀) alkenylene or alkyloxy, and bicyclic-fragments of 1,4:3,6-dianhydrohexitols of general formula (II): 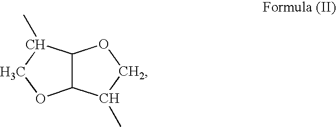
except that for unsaturated polymers having the chemical structure of structural formula (I), R¹ and R⁴ are selected from (C₂-C₂₀) alkylene and (C₂-C₂₀) alkenylene; wherein at least one of R¹ and R⁴ is (C₂-C₂₀) alkenylene; n is about 5 to about 150; each R² is independently hydrogen, or (C₆-C₁₀)aryl(C₁-C₆)alkyl; and each R³ is independently hydrogen, (C₁-C₆)alkyl, (C₂-C₆)alkenyl, (C₂-C₆)alkynyl, or (C₆-C₁₀)aryl(C₁-C₆)alkyl;
or a PEUR having a chemical formula described by general structural formula (III), 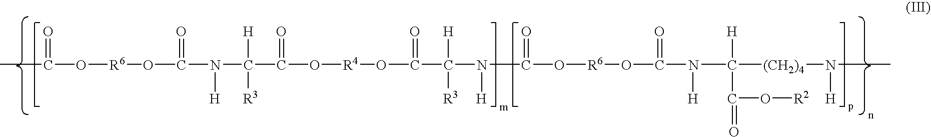
   and wherein n ranges from about 5 to about 150, m ranges about 0.1 to about 0.9: p ranges from about 0.9 to about 0.1; wherein R² is hydrogen or (C₆-C₁₀)aryl(C₁-C₆) alkyl or a protecting group, such as t-butyl; R³ is selected from the group consisting of hydrogen, (C₁-C₆) alkyl, (C₂-C₆) alkenyl, (C₂-C₆) alkynyl and (C₆-C₁₀) aryl(C₁-C₆) alkyl; R⁴ is selected from the group consisting of (C₂-C₂₀) alkylene, (C₂-C₂₀) alkenylene or alkyloxy, and bicyclic-fragments of 1,4:3,6-dianhydrohexitols of general formula (II); and R⁶ is independently selected from (C₂-C₂₀) alkylene, (C₂-C₂₀) alkenylene or alkyloxy, and bicyclic-fragments of 1,4:3,6-dianhydrohexitols of general formula (II). The bicyclic-fragments of such dianhydrohexitols can be derived from sugar alcohols, such as D-glucitol, D-mannitol and L-iditol.

In one alternative, R₃ is CH₂Ph and the alpha amino acid used in synthesis is L-phenylalanine. In alternatives wherein R₃ is CH₂—CH(CH₃)₂, the polymer contains the alpha-amino acid, leucine. By varying R₃, other alpha amino acids can also be used, e.g., glycine (when R₃ is H), alanine (when R₃ is CH₃), valine (when R₃ is CH(CH₃)₂), isoleucine (when R₃ is CH(CH₃—CH₂—CH₃), phenylalanine (when R₃ is CH₂—C₆H₅), or lysine (when R₃═(CH₂)₄—NH₂).

The polymer molecules may also have the bioactive agent conjugated thereto via a linker or incorporated into a crosslinker between molecules. For example, in one embodiment, the polymer is contained in a polymer-bioactive agent conjugate having the structural formula (IV): 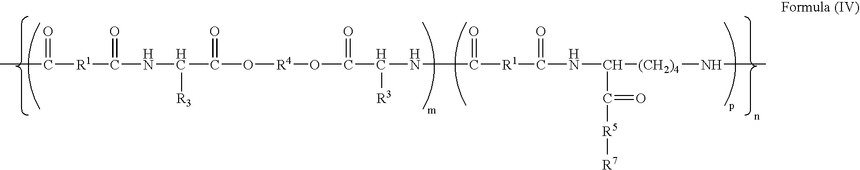
wherein n, m, p, R¹, R³, and R⁴ are as above, R⁵ is selected from the group consisting of —O—, —S—, and —NR⁸—, and wherein R⁸ is H or (C₁-C₈) alkyl; and R⁷ is the bioactive agent.

In yet another embodiment, two molecules of the polymer of structural formula (IV) can be crosslinked to provide an —R⁵—R⁷—R⁵— conjugate. In another embodiment, as shown in structural formula V below, the bioactive agent is covalently linked to two parts of a single polymer molecule of structural formula IV through the —R⁵—R⁷—R⁵— conjugate and R⁵ is independently selected from the group consisting of —O—, —S—, and —NR⁸—, wherein R⁸ is H or (C₁-C₈) alkyl; and R⁷ is the bioactive agent.

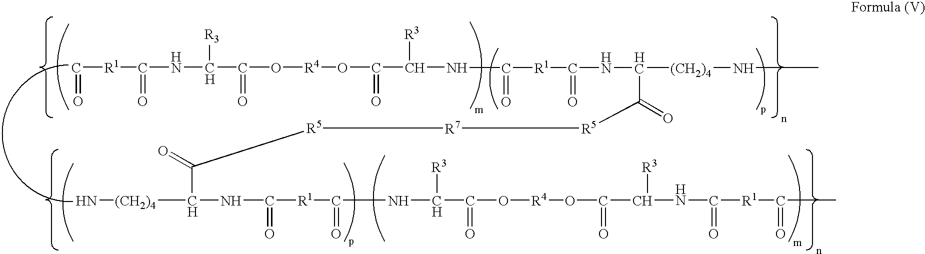

Alternatively still, as shown in structural formula (VI) below, a linker, —X—Y—, can be inserted between R⁵ and bioactive agent R⁷, in the molecule of structural formula (IV), wherein X is selected from the group consisting of (C₁-C₁₈) alkylene, substituted alkylene, (C₃-C₈) cycloalkylene, substituted cycloalkylene, 5-6 membered heterocyclic system containing 1-3 heteroatoms selected from the group O, N, and S, substituted heterocyclic, (C₂-C₁₈) alkenyl, substituted alkenyl, alkynyl, substituted alkynyl, C₆ and C₁₀ aryl, substituted aryl, heteroaryl, substituted heteroaryl, alkylaryl, substituted alkylaryl, arylalkynyl, substituted arylalkynyl, arylalkenyl, substituted arylalkenyl, arylalkynyl, substituted arylalkynyl and wherein the substituents are selected from the group H, F, Cl, Br, I, (C₁-C₆) alkyl, —CN, —NO₂, —OH, —O(C₁-C₄) alkyl), —S(C₁-C₆) alkyl), —S[(═O)(C₁-C₆) alkyl)], —S[(O₂)(C₁-C₆) alkyl], —C[(═O)(C₁-C₆) alkyl], CF₃, —O[(CO)—(C₁-C₆) alkyl)], —S(O₂)[N(R⁹R¹⁰), —NH[(C═O)(C₁-C₆) alkyl], —NH(C═O)N(R⁹R¹⁰), —N(R⁹R¹⁰); wherein R⁹ and R¹⁰ are independently H or (C₁-C₆)alkyl; and Y is selected from the group consisting of —O—, —S—, —S—S—, —S(O)—, —S(O₂)—, —NR⁸—, —C(═O)—, —OC(═O)—, —C(═O)O—, —OC(═O)NH—, —NR⁸C(═O)—, —C(═O)NR⁸—, —NR⁸C(═O)NR⁸—, —NR⁸C(═O)NR⁸—, and —NR⁸C(═S)NR⁸—.

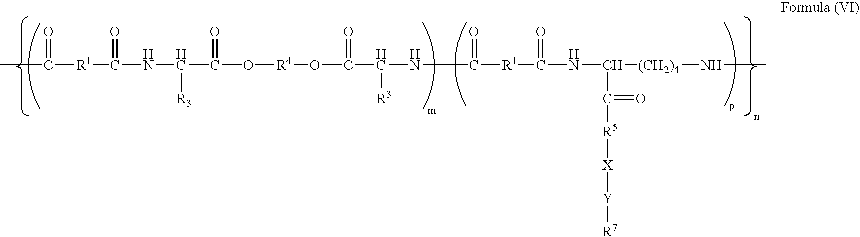

In another embodiment, two parts of a single macromolecule of structural formula (IV) are covalently linked to the bioactive agent through an —R⁵—R⁷—Y—X—R⁵— bridge (Formula VII): 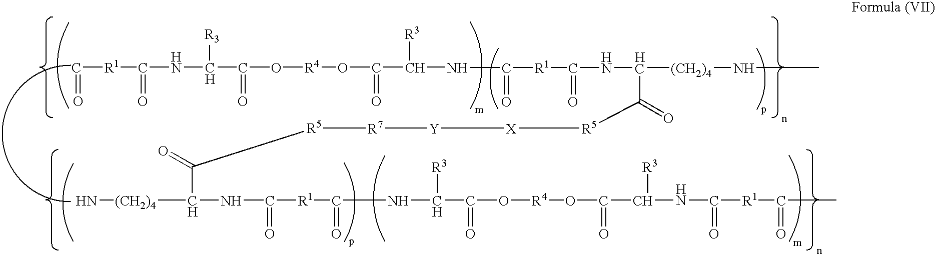
wherein, X is selected from the group consisting of (C₁-C₁₈) alkylene, substituted alkylene, (C₃-C₈) cycloalkylene, substituted cycloalkylene, 5-6 membered heterocyclic system containing 1-3 heteroatoms selected from the group O, N, and S, substituted heterocyclic, (C₂-C₁₈) alkenyl, substituted alkenyl, alkynyl, substituted alkynyl, (C₆-C₁₀) aryl, substituted aryl, heteroaryl, substituted heteroaryl, alkylaryl, substituted alkylaryl, arylalkynyl, substituted arylalkynyl, arylalkenyl, substituted arylalkenyl, arylalkynyl, substituted arylalkynyl, wherein the substituents are selected from the group consisting of H, F, Cl, Br, I, (C₁-C₆) alkyl, —CN, —NO₂, —OH, —O(C₁-C₆)alkyl), —S(C₁-C₆)alkyl), —S[(═O)(C₁-C₆ alkyl)], —S[(O₂)(C₁-C₆) alkyl], —C[(═O)(C₁-C₆) alkyl], CF₃, —O[(CO)—(C₁-C₆)alkyl)], —S(O₂)[N(R⁹R¹⁰), —NH[(C═O)(C₁-C₆)alkyl], —NH(C═O)N(R⁹R¹⁰), wherein R⁹ and R¹⁰ are independently H or (C₁-C⁶) alkyl and —N(R¹¹R¹²), wherein R¹¹ and R¹² are independently selected from (C₂-C₂₀) alkylene and (C₂-C₂₀) alkenylene.

In yet another embodiment, the polymer contains four molecules of the polymer of structural formula (IV), except that only two of the four molecules omit R⁷ and are crosslinked to provide a single —R⁵—X—R⁵— conjugate, wherein X is selected from the group consisting of (C₁-C₁₈) alkylene, substituted alkylene, (C₃-C₈) cycloalkylene, substituted cycloalkylene, 5-6 membered heterocyclic system containing 1-3 heteroatoms selected from the group O, N, and S, substituted heterocyclic, (C₂-C₁₈) alkenyl, substituted alkenyl, alkynyl, substituted alkynyl, C₆ and C₁₀ aryl, substituted aryl, heteroaryl, substituted heteroaryl, alkylaryl, substituted alkylaryl, arylalkynyl, substituted arylalkynyl, arylalkenyl, substituted arylalkenyl, arylalkynyl, substituted arylalkynyl and wherein the substituents are selected from the group consisting of H, F, Cl, Br, I, (C₁-C₆) alkyl, —CN, —NO₂.—OH, —O(C₁-C₄) alkyl), —S(C₁-C₆) alkyl), —S[(═O)(C₁-C₆) alkyl)], —S[(O₂)(C₁-C₆) alkyl], —C[(═O)(C₁-C₆) alkyl], —CF₃, —O[(CO)—(C₁-C₆) alkyl)], —S(O₂)[N(R⁹R¹⁰), —NH[(C═O)(C₁-C₆) alkyl], —NH(C═O)N(R⁹R¹⁰), and —N(R⁹R¹⁰); wherein R⁹ and R¹⁰ are independently H or C₁-C₆ alkyl).

In still another embodiment, four molecules of the polymer of structural formula III can be partially crosslinked by omitting the additional bioactive agent R₇ on two of the molecules and forming instead a single —R₅—X—R₅— conjugate, wherein X, R₅, and R₇ are as described above.

Further examples of PEA and PEUR polymers contemplated for use in the practice of the invention and methods of synthesis include those set forth in U.S. Pat. Nos. 5,516,881; 5,610,241, 6,338,047; 6,476,204; 6,503,538; and in U.S. application Ser. Nos. 10/096,435; 10/101,408; 10/143,572; 10/194,965 and 10/362,848.

These biodegradable polymers and copolymers preferably have weight average molecular weights ranging from 10,000 to 125,000; these polymers and copolymers typically have inherent viscosities at 25° C., determined by standard viscosimetric methods, ranging from 0.3 to 4.0, preferably ranging from 0.5 to 3.5.

The term “aryl” is used with reference to structural formulas herein to denote a phenyl radical or an ortho-fused bicyclic carbocyclic radical having about nine to ten ring atoms in which at least one ring is aromatic. In certain embodiments, one or more of the ring atoms can be substituted with one or more of nitro, cyano, halo, trifluoromethyl, or trifluoromethoxy. Examples of aryl include, but are not limited to, phenyl, naphthyl, and nitrophenyl.

The term “alkenylene” is used with reference to structural formulas herein to mean a divalent branched or unbranched hydrocarbon chain containing at least one unsaturated bond in the main chain or in a side chain.

The molecular weights and polydispersities herein are determined by gel permeation chromatography (GPC) using polystyrene standards. More particularly, number and weight average molecular weights (Mₙ and M_{w}) are determined, for example, using a Model 510 gel permeation chromatography (Water Associates, Inc., Milford, Mass.) equipped with a high-pressure liquid chromatographic pump, a Waters 486 UV detector and a Waters 2410 differential refractive index detector. Tetrahydrofuran (THF) is used as the eluent (1.0 mL/min). The polystyrene standards have a narrow molecular weight distribution.

Methods for making the polymers of formulas (I) and (III), containing an α-amino acid in the general formula are well known in the art. For example, for the embodiment of the polymer of formula (I), wherein the α-amino acid can be converted into a bis(α-amino acid) diester monomer, for example, by condensing the α-amino acid with a diol HOR⁴—OH. As a result, ester bonds are formed. Then, the bis(α-amino acid) diester is entered into a polycondensation reaction with a di-acid, such as sebacic acid, to obtain the final polymer having both ester and amide bonds. Alternatively, instead of the di-acid, an activated di-acid derivative, e.g., bis-para-nitrophenyl diester, can be used as an activated di-acid, for polymers of chemical structure (I) and (II)). Additionally, a bis-carbonate, such as bis(p-nitrophenyl) dicarbonate, can be used as the activated species to obtain polymers of structure (III). In the case of (III), a final polymer is obtained having both ester and urethane bonds.

More particularly, synthesis of the unsaturated poly(ester-amide)s (UPEAs) useful as biodegradable polymers of the structure (I) as described above will be described, wherein 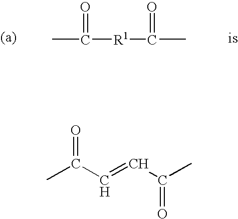
and/or (b) R⁴ is —CH₂—CH═CH—CH₂—. In cases where (a) is present and (b) is not present, R⁴ in (I) is —C₄H₈— or —C₆H₁₂—. In cases where (a) is not present and (b) is present, R¹ in (I) is —C₄H₈— or —C₈H₁₆—.

The UPEAs can be prepared by solution polycondensation of either (1) di-p-toluene sulfonic acid salt of bis(alpha-amino acid) diester of unsaturated diol and di-p-nitrophenyl ester of saturated dicarboxylic acid or (2) di-p-toluene sulfonic acid salt of bis(alpha-amino acid) diester of saturated diol and di-nitrophenyl ester of unsaturated dicarboxylic acid or (3) di-p-toluene sulfonic acid salt of bis(alpha-amino acid) diester of unsaturated diol and di-nitrophenyl ester of unsaturated dicarboxylic acid.

Salts of p-toluene sulfonic acid are known for use in synthesizing polymers containing amino acid residues. The aryl sulfonic acid salts are used instead of the free base because the aryl sulfonic salts of bis (alpha-amino acid) diesters are easily purified through recrystallization and render the amino groups as unreactive ammonium tosylates throughout workup. In the polycondensation reaction, the nucleophilic amino group is readily revealed through the addition of an organic base, such as triethylamine, so the polymer product is obtained in high yield.

For polymers of structure (I), the di-p-nitrophenyl esters of unsaturated dicarboxylic acid can be synthesized from p-nitrophenyl and unsaturated dicarboxylic acid chloride, e.g., by dissolving triethylamine and p-nitrophenol in acetone and adding unsaturated dicarboxylic acid chloride dropwise with stirring at −78° C. and pouring into water to precipitate product. Suitable acid chlorides included fumaric, maleic, mesaconic, citraconic, glutaconic, itaconic, ethenyl-butane dioic and 2-propenyl-butanedioic acid chlorides. For polymers of structure (III), bis-p-nitrophenyl dicarbonates of saturated or unsaturated diols are used as the activated monomer. Dicarbonate monomers of general structure (IX) are employed for polymers of structure (III) 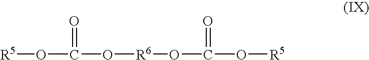
wherein each R⁵ is independently (C₆-C₁₀)aryl optionally substituted with one or more nitro, cyano, halo, trifluoromethyl, or trifluoromethoxy; and R⁶ is independently (C₂-C₂₀)alkylene or (C₂-C₂₀) alkyloxy, or (C₂-C₂₀)alkenylene.

The di-aryl sulfonic acid salts of diesters of alpha-amino acid and unsaturated diol can be prepared by admixing alpha-amino acid, e.g., p-aryl sulfonic acid monohydrate and saturated or unsaturated diol in toluene, heating to reflux temperature, until water evolution is minimal, then cooling. The unsaturated diols include, for example, 2-butene-1,3-diol and 1,18-octadec-9-en-diol.

Saturated di-p-nitrophenyl esters of dicarboxylic acid and saturated di-p-toluene sulfonic acid salts of bis-alpha-amino acid esters can be prepared as described in U.S. Pat. No. 6,503,538 B1.

Synthesis of the unsaturated poly(ester-amide)s (UPEAs) useful as biodegradable polymers of the structure (I) as described above will now be described. Compounds having the structure (I) can be made in similar fashion to the compound (VII) of U.S. Pat. No. 6,503,538 B1, except that R⁴ of (III) of U.S. Pat. No. 6,503,538 and/or R¹ of (V) of U.S. Pat. No. 6,503,538 is C₂-C₂₀ alkenylene as described above. The reaction is carried out, for example, by adding dry triethylamine to a mixture of said (III) and (IV) of U.S. Pat. No. 6,503,538 and said (V) of U.S. Pat. No. 6,503,538 in dry N,N-dimethylacetamide, at room temperature, then increasing the temperature to 80° C. and stirring for 16 hours, then cooling the reaction solution to room temperature, diluting with ethanol, pouring into water, separating polymer, washing separated polymer with water, drying to about 30° C. under reduced pressure and then purifying up to negative test on p-nitrophenyl and p-toluene sulfonic acid. A preferred reactant (IV) of U.S. Pat. No. 6,503,538 is p-toluene sulfonic acid salt of benzyl ester, the benzyl ester protecting group is preferably removed from (II) to confer biodegradability, but it should not be removed by hydrogenolysis as in Example 22 of U.S. Pat. No. 6,503,538 because hydrogenolysis would saturate the desired double bonds; rather the benzyl ester group should be converted to an acid group by a method that would preserve unsaturation, e.g., by treatment with fluoroacetic acid or gaseous HF. Alternatively, the lysine reactant (IV) of U.S. Pat. No. 6,503,538 can be protected by a protecting group different from benzyl which can be readily removed in the finished product while preserving unsaturation, e.g., the lysine reactant can be protected with t-butyl (i.e., the reactant can be t-butyl ester of lysine) and the t-butyl can be converted to H while preserving unsaturation by treatment of the product (II) with acid.

A working example of the compound having structural formula (I) is provided by substituting p-toluene sulfonic acid salt of bis(L-phenylalanine) 2-butene-1,4-diester for (III) in Example 1 of U.S. Pat. No. 6,503,538 or by substituting di-p-nitrophenyl fumarate for (V) in Example 1 of U.S. Pat. No. 6,503,538 or by substituting the p-toluene sulfonic acid salt of bis(L-phenylalanine) 2-butene-1,4-diester for III in Example 1 of U.S. Pat. No. 6,503,538 and also substituting bis-p-nitrophenyl fumarate for (V) in Example 1 of U.S. Pat. No. 6,503,538.

In unsaturated compounds having structural formula (I), the following hold: An amino substituted aminoxyl (N-oxide) radical bearing group e.g., 4-amino TEMPO, can be attached using carbonyldiimidazole as a condensing agent. Wound healing agents and additional bioactive agents, and the like, as described herein, can be attached via the double bond functionality. Hydrophilicity can be imparted by bonding to poly(ethylene glycol) diacrylate.

The biodegradable polymers and copolymers preferably have weight average molecular weights ranging from 10,000 to 300,000; these polymers and copolymers typically have intrinsic viscosities at 25° C., determined by standard viscosimetric methods, ranging from 0.3 to 4.0, preferably ranging from 0.5 to 3.5.

Polymers contemplated for use in the practice of the invention can be synthesized by a variety of methods well known in the art. For example, tributyltin (IV) catalysts are commonly used to form polyesters such as poly(caprolactone), poly(glycolide), poly(lactide), and the like. However, it is understood that a wide variety of catalysts can be used to form polymers suitable for use in the practice of the invention.

Such poly(caprolactones) contemplated for use have an exemplary structural formula (VIII) as follows:

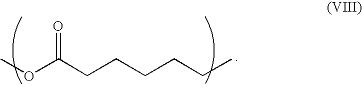

Poly(glycolides) contemplated for use have an exemplary structural formula (IX) as follows:

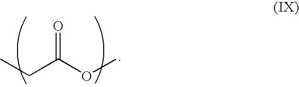

Poly(lactides) contemplated for use have an exemplary structural formula (X) as follows:

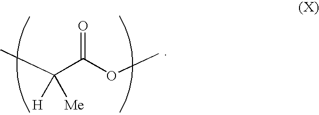

An exemplary synthesis of a suitable poly(lactide-co-ε-caprolactone) including an aminoxyl moiety is set forth as follows. The first step involves the copolymerization of lactide and ε-caprolactone in the presence of benzyl alcohol using stannous octoate as the catalyst to form a polymer of structural formula (XI).

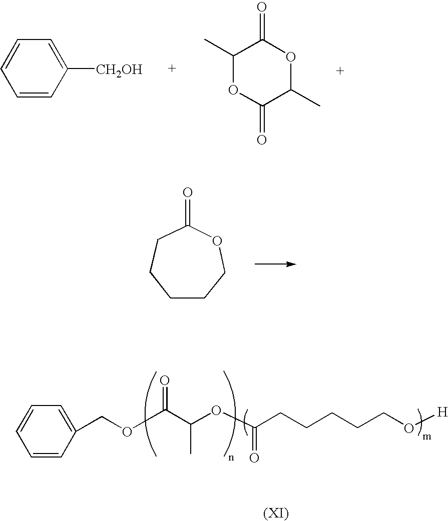

The hydroxy terminated polymer chains can then be capped with maleic anhydride to form polymer chains having structural formula (XII):

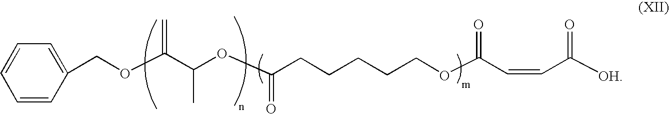

At this point, 4-amino-2,2,6,6-tetramethylpiperidine-1-oxy can be reacted with the carboxylic end group to covalently attach the aminoxyl moiety to the copolymer via the amide bond which results from the reaction between the 4-amino group and the carboxylic acid end group. Alternatively, the maleic acid capped copolymer can be grafted with polyacrylic acid to provide additional carboxylic acid moieties for subsequent attachment of further aminoxyl groups.

In certain embodiments, the bioactive agent can be covalently bound to the biodegradable polymers via a wide variety of suitable functional groups. For example, when the biodegradable polymer is a polyester, the carboxyl group chain end can be used to react with a complimentary moiety on the bioactive agent, such as hydroxy, amino, thio, and the like. A wide variety of suitable reagents and reaction conditions are disclosed, e.g., in Advanced Organic Chemistry, Reactions, Mechanisms, and Structure, Fifth Edition, (2001); and Comprehensive Organic Transformations, Second Edition, Larock (1999).

In other embodiments, a bioactive agent can be dispersed into the polymer by “loading” onto the polymer without formation of a chemical bond or the bioactive agent can be linked to any of functional group in the polymers, such as an amide, ester, ether, amino, ketone, thioether, sulfinyl, sulfonyl, disulfide, and the like, to form a direct linkage. Such a linkage can be formed from suitably functionalized starting materials using synthetic procedures that are known in the art.

For example, a polymer of the present invention can be linked to the bioactive agent via a carboxyl group (e.g., COOH) of the polymer. Specifically, a compound of structures (I)-(VI) can react with an amino functional group of a bioactive agent or a hydroxyl functional group of a bioactive agent to provide a biodegradable, biocompatible polymer having the bioactive agent attached via an amide linkage or carboxylic ester linkage, respectively. In another embodiment, the carboxyl group of the polymer can be transformed into an acyl halide, acyl anhydride/“mixed” anhydride, or active ester.

Alternatively, the bioactive agent may be attached to the polymer via a linker. Indeed, to improve surface hydrophobicity of the biodegradable polymer, to improve accessibility of the biodegradable polymer towards enzyme activation, and to improve the release profile of the biodegradable polymer, a linker may be utilized to indirectly attach the bioactive agent to the biodegradable polymer. In certain embodiments, the linker compounds include poly(ethylene glycol) having a molecular weight (MW) of about 44 to about 10,000, preferably 44 to 2000; amino acids, such as serine; polypeptides with repeat units from 1 to 100; and any other suitable low molecular weight polymers. The linker typically separates the bioactive agent from the polymer by about 5 angstroms up to about 200 angstroms.

In still further embodiments, the linker is a divalent radical of formula W-A-Q, wherein A is (C₁-C₂₄)alkyl, (C₂-C₂₄)alkenyl, (C₂-C₂₄)alkynyl, (C₃-C₈)cycloalkyl, or (C₆-C₁₀) aryl, and W and Q are each independently —N(R)C(═O)—, —C(═O)N(R)—, —OC(═O)—, —C(═O)O, —O—, —S—, —S(O), —S(O)₂—, —S—S—, —N(R)—, —C(═O)—, wherein each R is independently H or (C₁-C₆)alkyl.

As used herein, the term “alkyl” refers to a straight or branched chain hydrocarbon group including methyl, ethyl, n-propyl, isopropyl, n-butyl, isobutyl, tert-butyl, n-hexyl, and the like.

As used herein, “alkenyl” refers to straight or branched chain hydrocarbon groups having one or more carbon-carbon double bonds.

As used herein, “alkynyl” refers to straight or branched chain hydrocarbon groups having at least one carbon-carbon triple bond.

As used herein, “aryl” refers to aromatic groups having in the range of 6 up to 14 carbon atoms.

In certain embodiments, the linker may be a polypeptide having from about 2 up to about 25 amino acids. Suitable peptides contemplated for use include poly-L-lysine, poly-L-glutamic acid, poly-L-aspartic acid, poly-L-histidine, poly-L-ornithine, poly-L-threonine, poly-L-tyrosine, poly-L-leucine, poly-L-lysine-L-phenylalanine, poly-L-arginine, poly-L-lysine-L-tyrosine, and the like.

The linker can be attached first to the polymer or to the bioactive agent. During synthesis of polymers containing bioactive agents indirectly attached via a linker, the linker can be either in unprotected form or protected from, using a variety of protecting groups well known to those skilled in the art.

In the case of a protected linker, the unprotected end of the linker can first be attached to the polymer or the bioactive agent. The protecting group can then be de-protected using Pd/H₂ hydrogenolysis, mild acid or base hydrolysis, or any other common de-protection method that are known in the art. The de-protected linker can then be attached to the bioactive agent. An example using poly(ethylene glycol) as the linker is shown in Scheme 1.

Scheme 1

Poly(ethylene glycol) employed as the linker between polymer and bioactive or additional bioactive agent. 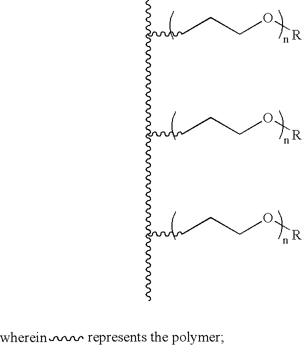
R can be either a drug or bioactive agent; and
n can range from 1 to 200; preferable from 1 to 50.

An exemplary synthesis of a biodegradable, bioactive polymer according to the invention (wherein the bioactive agent is an aminoxyl) is set forth as follows. A polyester can be reacted with an amino substituted aminoxyl (N-oxide) radical bearing group, e.g., 4-amino-2,2,6,6-tetramethylpiperidine-1-oxy, in the presence of N,N′-carbonyldiimidazole to replace the hydroxyl moiety in the carboxyl group at the chain end of the polyester with an amino substituted aminoxy]-(N-oxide) radical bearing group, so that the amino moiety covalently bonds to the carbon of the carbonyl residue of the carboxyl group to form an amide bond. The N,N′-carbonyldiimidazole or suitable carbodiimide converts the hydroxyl moiety in the carboxyl group at the chain end of the polyester into an intermediate product moiety which will react with the aminoxyl, e.g., 4-amino-2,2,6,6-tetramethylpiperidine-1-oxy. The aminoxyl reactant is typically used in a mole ratio of reactant to polyester ranging from 1:1 to 100:1. The mole ratio of N,N′-carbonyldiimidazole to aminoxyl is preferably about 1:1.

A typical reaction is as follows. A polyester is dissolved in a reaction solvent and reaction is readily carried out at the temperature utilized for the dissolving. The reaction solvent may be any in which the polyester will dissolve; this information is normally available from the manufacturer of the polyester. When the polyester is a polyglycolic acid or a poly(glycolide-L-lactide) (having a monomer mole ratio of glycolic acid to L-lactic acid greater than 50:50), highly refined (99.9+% pure) dimethyl sulfoxide at 115° C. to 130° C. or hexafluoroisopropanol at room temperature suitably dissolves the polyester. When the polyester is a poly-L-lactic acid, a poly-DL-lactic acid or a poly(glycolide-L-lactide) (having a monomer mole ratio of glycolic acid to L-lactic acid 50:50 or less than 50:50), tetrahydrofuran, methylene chloride and chloroform at room temperature to 50° C. suitably dissolve the polyester.

The reaction is typically carried out to substantial completion in 30 minutes to 5 hours. When a polyglycolic acid or a poly(glycolide-L-lactide) from a glycol-rich monomer mixture constitutes the polyester, 2 to 3 hours of reaction time is preferred. When a poly-L-lactic acid is the polyester, the reaction is readily carried out to substantial completion at room temperature for one hour. The reaction is preferably carried out under an inert atmosphere with dry nitrogen purging so as to drive the reaction towards completion.

The product may be precipitated from the reaction mixture by adding cold non-solvent for the product. For example, aminoxy]-containing polyglycolic acid and aminoxy]-containing poly(glycolide-L-lactide) formed from glycolic acid-rich monomer mixture are readily precipitated from hot dimethylsulfoxide by adding cold methanol or cold acetone/methanol mixture and then recovered, e.g., by filtering. When the product is not readily precipitated by adding cold non-solvent for the product, the product and solvent may be separated by using vacuum techniques. For example, aminoxyl-containing poly-L-lactic acid is advantageously separated from solvent in this way. The recovered product is readily further purified by washing away water and by-products (e.g. urea) with a solvent which does not dissolve the product, e.g., methanol in the case of the modified polyglycolic acid, polylactic acid and poly(glycolide-L-lactide) products herein. Residual solvent from such washing may be removed using vacuum drying.

The polymers described herein can be coated onto the surface of a surgical device as described here in many ways, such as dip-coating, spray-coating, ionic deposition, and the like, as is well known in the art. In coating a porous surface of a surgical device, care must be taken not to occlude the pores, which are needed to allow access and migration of cells, factors, and the like, from the surface of the device to the interior of the device, for example endothelial cells and other blood factors that participate in the natural biological process of wound healing.

The surgical device, with at least a portion of its surface coated with the biodegradable polymer(s) into which the bioactive agent is dispersed, can be formed of any suitable substance, such as is known in the art. For example, the surgical device can be formed from a biocompatible metal, such as stainless steel, tantalum, nitinol, elgiloy, and the like, and suitable combinations thereof. For porous surgical devices, such as stents, the biocompatible material is selected to be molded, stamped, or woven, and the like, to contain the porous surface features described herein. For example, the surgical device can itself be substantially biodegradable, being made of cross-linkable “star structure polymers”, or dendrimers, which are well known to those skilled in the art. In one aspect, the surgical device is formed from biodegradable cross-linked poly(ester amide), polycaprolactone, or poly(ester urethane) as described herein.

Polymer/Bioactive Agent Linkage

In one embodiment, the polymers used to make the wound dressings and device coverings as described herein have one or more bioactive agents that promote natural re-endothelialization of vessels directly linked to the polymer. The residues of the polymer can be linked to the residues of the one or more bioactive agents. For example, one residue of the polymer can be directly linked to one residue of the bioactive agent. The polymer and the bioactive agent can each have one open valence. Alternatively, more than one bioactive agent, or a mixture of bioactive agents, that promote natural re-endothelialization of vessels can be directly linked to the polymer. However, since the residue of each bioactive agent can be linked to a corresponding residue of the polymer, the number of residues of the one or more bioactive agents can correspond to the number of open valences on the residue of the polymer.

As used herein, a “residue of a polymer” refers to a radical of a polymer having one or more open valences. Any synthetically feasible atom, atoms, or functional group of the polymer (e.g., on the polymer backbone or pendant group) of the present invention can be removed to provide the open valence, provided bioactivity is substantially retained when the radical is attached to a residue of a bioactive agent. Additionally, any synthetically feasible functional group (e.g., carboxyl) can be created on the polymer (e.g., on the polymer backbone or pendant group) to provide the open valence, provided bioactivity is substantially retained when the radical is attached to a residue of a bioactive agent. Based on the linkage that is desired, those skilled in the art can select suitably functionalized starting materials that can be derived from the polymer of the present invention using procedures that are known in the art. As used herein, a “residue of a compound of formula (*)” refers to a radical of a compound of formulas (I-VI) having one or more open valences. Any synthetically feasible atom, atoms, or functional group of the compound of formulas (I-VI) (e.g., on the polymer backbone or pendant group) can be removed to provide the open valence, provided bioactivity is substantially retained when the radical is attached to a residue of a bioactive agent. Additionally, any synthetically feasible functional group (e.g., carboxyl) can be created on the compound of formulas (I-VI) (e.g., on the polymer backbone or pendant group) to provide the open valance, provided bioactivity is substantially retained when the radical is attached to a residue of a bioactive agent. Based on the linkage that is desired, those skilled in the art can select suitably functionalized starting materials that can be derived from the compound of formulas (I-VI) using procedures that are known in the art.

The residue of a bioactive agent can be linked to the residue of a compound of formula (I)-(VI) through an amide (e.g., —N(R)C(═O)— or —C(═O)N(R)—), ester (e.g., —OC(═O)— or —C(═O)O—), ether (e.g., —O—), amino (e.g., —N(R)—), ketone (e.g., —C(═O)—), thioether (e.g., —S—), sulfinyl (e.g., —S(O)—), sulfonyl (e.g., —S(O)₂—), disulfide (e.g., —S—S—), or a direct (e.g., C—C bond) linkage, wherein each R is independently H or (C—C₆) alkyl. Such a linkage can be formed from suitably functionalized starting materials using synthetic procedures that are known in the art. Based on the linkage that is desired, those skilled in the art can select suitably functional starting materials that can be derived from a residue of a compound of formula (I)-(VI) and from a given residue of a bioactive agent using procedures that are known in the art. The residue of the bioactive agent can be directly linked to any synthetically feasible position on the residue of a compound of formulas (I-VI). Additionally, the invention also provides compounds having more than one residue of a bioactive agent or bioactive agents directly linked to a compound of formulas (I-VI).

One or more bioactive agents can be linked directly to the polymer. Specifically, the residue of each of the bioactive agents can each be directly linked to the residue of the polymer. Any suitable number of bioactive agents (i.e., residues thereof) can be directly linked to the polymer (i.e., residue thereof) either through a functional group or through a double or triple bond. The number of bioactive agents that can be directly linked to the polymer can typically depend upon the molecular weight of the polymer and the number of its free functional groups and double or triple bonds. For example, for a saturated compound of formula (I), wherein n is about 50 to about 150, up to about 300 bioactive agents (i.e., residues thereof) can be directly linked to the polymer (i.e., residue thereof) by reacting the bioactive agent with end groups of the polymer. Suitable reagents and reaction conditions for creating such linkages are disclosed, e.g., in Advanced Organic Chemistry, Part B: Reactions and Synthesis, Second Edition, Carey and Sundberg (1983); Advanced Organic Chemistry, Reactions, Mechanisms, and Structure, Second Edition, March (1977); and Comprehensive Organic Tiansformations, Second Edition, Larock (1999).

In one embodiment of the present invention, a polymer (i.e., residue thereof) can be linked to the bioactive agent (i.e., residue thereof) via the carboxyl group (e.g., COOR²) of the polymer. Specifically, a compound of formula (I) wherein R is independently hydrogen, or (C₆-C₁₀) aryl (C₁₋C₆)alkyl; can react with an amino functional group of a bioactive agent or a hydroxyl functional group of a bioactive agent, to provide a Polymer/Bioactive agent having an amide linkage or a Polymer/Bioactive agent having a carboxylic ester linkage, respectively. In another embodiment, the carboxyl group of the polymer can be transformed into an acyl halide or an acyl anhydride.

Hydrogels for Use in Wound Healing

Non-stick wound healing dressings and non-stick layers used in the invention wound-healing dressings and implantable drug delivery compositions comprise a biodegradable hydrogel. Although any biodegradable hydrogel known in the art that can be loaded with a wound healing drug or agent for in situ delivery can be used for this purpose, preferred hydrogels have both hydrophobic and hydrophilic components and form a one-phase crosslinked polymer network structure by free radical polymerization. Such hydrogels effectively accommodate hydrophobic drugs (as well as hydrophilic drugs) and hydrogels with hydrophobic and hydrophilic components have the advantage of maintaining structural integrity for relatively longer periods of time and having increased mechanical strength compared to totally hydrophilic-based hydrogels. Due to its non-stick nature, the hydrogel layer can be placed directly into the wound bed to deliver its load of least one wound-healing bioactive agent (i.e., a bioactive agent that produces a wound healing effect) in situ and can be removed without damage to the developing wound healing structures in the wound bed.

In one aspect, such a hydrogel is formed from a hydrogel-forming system that comprises from 0.01 to 99.99% by weight, for example, from 95% to 5%, by weight of (A), wherein (A) is a hydrophobic macromer with unsaturated group terminated ends, and from 99.99 to 0.01% by weight, for example, from 5% to 95%, by weight of (B), wherein (B) is a hydrophilic polysaccharide containing hydroxyl groups that are reacted with the unsaturated groups of the hydrophobic macromer. The total of the percentages of (A) and (B) is 100%. The hydrophobic macromer is biodegradable and is readily prepared by reacting diol, obtained by converting hydroxyls of terminal carboxylic acid groups of poly(lactic acid) to amidoethanol groups, with an unsaturated group-introducing compound. For example, the unsaturated-group introducing compound may contain a carboxylic acid, which can be reacted with the terminal diol of the polymer to form ester bonds to the unsaturated group.

For example, the hydrophilic polymer can be dextran wherein one or more hydroxyls in a glucose unit of the dextran are reacted with the unsaturated group-introducing compound. In one case, the hydrophilic polymer can be dextran-maleic acid monoester as described in PCT/US99/18818, which is incorporated herein by reference.

A wound-healing bioactive agent or drug, as described herein, can be loaded into (i.e., dispersed in) the hydrogel by a number of means depending on the molecular weight of the agent or drug. For example, a drug of weight average molecular weight ranging from 200 to 1,000, as exemplified by indomethacin, can be entrapped in the three dimensional crosslinked polymer network for controlled release therefrom. Alternatively, a water-soluble macromolecule of weight average molecular weight ranging from 1,000 to 10,000, e.g., a polypeptide, as exemplified by insulin, can be entrapped in the three dimensional crosslinked polymer network for controlled release therefrom. In still another example, a synthetic or natural polymer, e.g., of weight average molecular weight ranging from 10,000 to 100,000, can be entrapped in the three dimensional crosslinked polymer network for controlled release therefrom.

The term “hydrogel” is used herein to mean a polymeric material that exhibits the ability to imbibe water and to retain a significant portion of the water within its structure without dissolving.

A “biodegradable hydrogel” as the term is used herein is a hydrogel formed from a hydrogel forming system containing at least one biodegradable component, i.e., a component that is degraded by water and/or by enzymes found in wounds of mammalian patients, such as humans. The invention wound dressings are also suitable for use in veterinary treatment of wounds in a variety of mammalian patients, such as pets (for example, cats, dogs, rabbits, ferrets), farm animals (for example, swine, horses, mules, dairy and meat cattle) and race horses.

The term “crosslinked polymer network structure” is used herein to mean an interconnected structure where crosslinks are formed between hydrophobic molecules, between hydrophilic molecules and between hydrophobic molecules and hydrophilic molecules.

The term “photocrosslinking” is used herein to mean the formation of new carbon-carbon bonds from vinyl bonds of two species, or from unsaturated moieties of two species, by the application of appropriate radiant energy. A photo initiator may be used to commence the photocrosslinking process, by providing a reactive free radical to initiate crosslinking upon application of the appropriate radiant energy, as is well known in the art.

The term “macromer” is used herein to mean a monomer having a weight average molecular weight ranging from 500 to 80,000.

The term “unsaturated group-introducing compound” is used herein with respect to hydrogels and means a compound that reacts with an hydroxyl group and provides a pendant or end group containing an unsaturated group, e.g., a pendant group with a vinyl group at its end.

The weight average molecular weights and number average molecular weights herein are determined by gel permeation chromatography.

A detailed description of such biodegradable hydrogels and their methods of preparation are described in U.S. Pat. Nos. 6,476,204, 6,388,047, 6,583,219, 6,716,445; in U.S. provisional Application No. 60/098,571, and in U.S. application Ser. Nos. 09/531,451, 10/096,435, 10/143,572, 10/362,848, and 10/369,676.

Suitable compounds for use as the hydrophobic macromer (A) used in the preparation of biodegradable hydrogels are readily obtained by converting the end groups of a starting material macromer to groups with terminal hydroxyl group if such are not already present as end groups, i.e., to provide a diol, and reacting the terminal hydroxyls with an unsaturated group-introducing compound to provide terminal unsaturated groups, e.g., vinyl groups, on the macromer. The starting material macromer preferably has a weight average molecular weight ranging up from 500 to 20,000, such as the aliphatic polyester poly(lactic acid) having a weight average molecular weight ranging from 600 to 8,000, e.g., 600 to 1,000 or 6,500 to 8,000, e.g., poly-D-,L-lactic acid (sometimes denoted PDLLA). Poly-D,L-lactic acid has widely been used as a biodegradable hydrophobic polymeric material due to its combination of biodegradability, biocompatibility, and adequate mechanical strength. The degradation of poly-D,L-lactic acid in vivo is well understood and the degradation products are natural metabolites that can be readily eliminated by the human body. Other starting material macromers that can be used include, for example, other aliphatic polyesters, such as poly(glycolic acid), poly(epsilon-caprolactone), poly(glycolide-co-lactide), poly(lactide-epsilon-caprolactone), polycaprolactone diols (e.g., with Mₙ equal to 530, 1250 or 2000), polycaprolactone triols (e.g., with Mₙ equal to 300 or 900), or any synthetic biodegradable macromer having one carboxyl end group and one hydroxyl end group, carboxyl groups at both ends, or hydroxyl groups at both ends.

Reaction of a diol with the unsaturated group-introducing compound provides a hydrophobic polymer with unsaturated end groups. The unsaturated group-introducing compound can be, for example, acryloyl chloride, methacryloyl chloride, acrylic acid, methacrylic acid, or isocyanate having unsaturated, e.g., vinyl, group at one end of the molecule, e.g., allyl isocyanate or isocyanatoethyl methacrylate. Vinyl terminated hydrophobic macromer A can be prepared from poly-D,L-lactic acid with mers ranging from 8 to 120.

The hydrophilic polymer (B) is a polysaccharide derivative. Suitable polysaccharides useful for preparing (B) have hydroxy functional pendant groups and include, for example, dextran, inulin, starch, cellulose, pullan, levan, mannan, chitin, xylan, pectin, glucuronan, laminarin, galactomannan, amylose, amylopectin, and phytoglucans. These polysaccharides have multiple hydroxy functional groups that permit the production of a three-dimensional network. The named polysaccharides are inexpensive. Dextran, which is the preferred polysaccharide starting material, is one of the most abundant naturally occurring biodegradable polymers. It is susceptible to enzymatic digestion in the body and consists mainly of (1→6) alpha-D-glucoside linkages with about 5-10% of (1→3) alpha-linked branching. It contains three hydroxyl groups per glucose repeating unit and therefore mediates formation of a crosslinked polymer network. Preferably, the dextran starting material has a weight average molecular weight ranging from 40,000 to 80,000.

The polysaccharide hydroxy groups are reacted with an unsaturated group-introducing compound. Suitable unsaturated group-introducing compounds for use in making biodegradable hydrogels include, for example, acryloyl chloride, methacryloyl chloride, acrylic acid, methacrylic acid, or isocyanate having an unsaturated, e.g., vinyl, group at one end of the molecule, e.g., allyl isocyanate or isocyanatoethyl methacrylate.

The percentages of (A) and (B), the molecular weight of the hydrophobic macromer, the molecular weight of the hydrophilic polymer, and the degree of substitution in the hydrophilic polymer, are variables affecting hydrophobicity/hydrophilicity, mechanical, swelling ratio and biodegradation properties of the hydrogel prepared from the hydrogel-forming systems described herein. The “swelling ratio” is obtained by immersing a known weight of dry hydrogel in a vial containing 15 ml liquid, removing swollen hydrogel from the liquid at regular time intervals wiping off surface water and weighing, until equilibrium is obtained.

Decreasing the percentage of (B) and increasing the percentage of (A) increases hydrophobicity (and compatibility with hydrophobic agents and milieus) and decreases swelling ratio (with the largest percentage decrease in swelling ratio being found in decreasing the percentage of (B) from 80% to 60% and increasing the percentage of (A) from 20% to 40%). Increasing the percentage of (B) and decreasing the percentage of (A) increases hydrophilicity and compatibility of hydrogel with hydrophilic agents and milieus. Increasing the percentage of (A) improved mechanical properties in the hydrogels formed from the hydrogel-forming systems. Increasing the molecular weight of (A) increases hydrophobicity and enhances mechanical properties, increases swelling ratio where the percentage of A or B is high and causes increase in biodegradation time for formed hydrogel. Increase in the molecular weight of (B) decreases hydrophobicity, decreases swelling ratio, enhances mechanical properties, and where (B) is a dextran derivative increases time for degradation by dextranase, in formed hydrogel. Increase in degree of substitution in hydrophilic polymer decreases hydrophilicity and swelling ratio (in higher weight percentage dextran derivative compositions), enhances mechanical properties and increases degradation time, in formed hydrogel.

The hydrogel formed herein can chemically incorporate a wound-healing bioactive agent which reacts with either or both of the components of the hydrogel-forming system; this can be accomplished by reacting the bioactive agent with one or both of the components of the hydrogel-forming system herein.

Wound-healing agents which are not reactive with components of the hydrogel-forming system herein can be physically entrapped within the hydrogel or physically encapsulated within the hydrogel by including them in the reaction mixture subjected to photocrosslinking so that the photocrosslinking causes formation of hydrogel with bioactive agent entrapped therein or encapsulated thereby.

By varying the parameters as discussed above, to vary mechanical properties, hydrophobicity/hydrophilicity, swelling ratio and biodegradation properties, the hydrogel-forming system described herein can be tailored to produce hydrogels for drug control release devices, for wound coverage, for coating surgical implants (e.g., for coating an artificial pancreas). As described above, higher swelling ratios give faster drug release and are connected with high hydrophilicity, which is important for wound cleaning utilities, and provide better absorption for sanitary purposes. The hydrogels of the invention herein are useful, for example, for the controlled release of low molecular weight drugs, water-soluble macromolecules and proteins as well as for the scaffolds for tissue engineering.

The synthetic or natural polymers that can be incorporated into biodegradable hydrogels include, for example, proteins, peptides, polysaccharides, and polymucosaccharides. Proteins for this alternative include, for example, lysozyme, interleukin-1, and basic fibroblast growth factor. This alternative provides a good approach for controlled release administration of synthetic or natural polymer drugs.

Entrapped wound-healing agents are readily incorporated into the biodegradable hydrogel by forming a solution of components (A) and (B) to provide a concentration of 30 to 50% (w/v) of total of (A) and (B) in the solution, adding photo initiator and then adding, for example, from 0.5 to 3% (w/w based on the total weight of (A) and (B)) of agent to be entrapped, and then effecting free radical polymerization. The solvent should be one in which (A) and (B), and agent to be entrapped are soluble. is used in the examples. Such solvents in which (A) and (B) are soluble typically include, for example, N,N-dimethylformamide (DMF) and dimethyl sulfoxide (DMSO), and selection is made from among the solvents in which (A) and (B) are soluble, to obtain solvent that also dissolves the agent to be entrapped.

Additional Bioactive Agents

As used herein, the term “additional bioactive agent” refers to a therapeutic, palliative, or diagnostic agent, other than the “wound healing agents” described above. Such additional bioactive agents can also be dispersed within a hydrogel or polymer matrix or coating on the surface of insertable or implantable surgical devices having different treatment aims as are known in the art, wherein release of the additional bioactive agent from the hydrogel or the polymer coating by biodegradation is desirable, for example, by contact with a treatment surface or blood borne cell or factor.

As used herein, the term “bioactive agent” is a general term used to refer to and encompass both wound healing agents and additional bioactive agents, as those terms are used herein, that can be incorporated into the polymers and/or hydrogels used in the invention compositions.

Specifically, such additional bioactive agents can include, but are not limited to, one or more of: polynucleotides, polypeptides, oligonucleotides, nucleotide analogs, nucleoside analogs, polynucleic acid decoys, therapeutic antibodies, abciximab, blood modifiers, anti-platelet agents, anti-coagulation agents, immune suppressive agents, anti-neoplastic agents, anti-cancer agents, anti-cell proliferation agents, and nitric oxide releasing agents.

The polynucleotide can include deoxyribonucleic acid (DNA), ribonucleic acid (RNA), double stranded DNA, double stranded RNA, duplex DNA/RNA, antisense polynucleotides, functional RNA or a combination thereof. In one embodiment, the polynucleotide can be RNA. In another embodiment, the polynucleotide can be DNA. In another embodiment, the polynucleotide can be an antisense polynucleotide. In another embodiment. the polynucleotide can be a sense polynucleotide. In another embodiment, the polynucleotide can include at least one nucleotide analog. In another embodiment, the polynucleotide can include a phosphodiester linked 3′-5′ and 5′-3′ polynucleotide backbone. Alternatively. the polynucleotide can include non-phosphodiester linkages, such as phosphotioate type, phosphoramidate and peptide-nucleotide backbones. In another embodiment, moieties can be linked to the backbone sugars of the polynucleotide. Methods of creating such linkages are well known to those of skill in the art.

The polynucleotide can be a single-stranded polynucleotide or a double-stranded polynucleotide. The polynucleotide can have any suitable length. Specifically, the polynucleotide can be about 2 to about 5,000 nucleotides in length, inclusive; about 2 to about 1000 nucleotides in length, inclusive; about 2 to about 100 nucleotides in length, inclusive; or about 2 to about 10 nucleotides in length, inclusive.

An antisense polynucleotide is typically a polynucleotide that is complimentary to an mRNA, which encodes a target protein. For example, the mRNA can encode a cancer promoting protein i.e., the product of an oncogene. The antisense polynucleotide is complimentary to the single-stranded mRNA and will form a duplex and thereby inhibit expression of the target gene, i.e., will inhibit expression of the oncogene. The antisense polynucleotides of the invention can form a duplex with the mRNA encoding a target protein and will disallow expression of the target protein.

A “functional RNA” refers to a ribozyme or other RNA that is not translated.

A “polynucleic acid decoy” is a polynucleic acid which inhibits the activity of a cellular factor upon binding of the cellular factor to the polynucleic acid decoy. The polynucleic acid decoy contains the binding site for the cellular factor. Examples of cellular factors include, but are not limited to, transcription factors, polymerases and ribosomes. An example of a polynucleic acid decoy for use as a transcription factor decoy will be a double-stranded polynucleic acid containing the binding site for the transcription factor. Alternatively, the polynucleic acid decoy for a transcription factor can be a single-stranded nucleic acid that hybridizes to itself to form a snap-back duplex containing the binding site for the target transcription factor. An example of a transcription factor decoy is the E2F decoy. E2F plays a role in transcription of genes that are involved with cell-cycle regulation and that cause cells to proliferate. Controlling E2F allows regulation of cellular proliferation. For example, after injury (e.g., angioplasty, surgery, stenting) smooth muscle cells proliferate in response to the injury. Proliferation may cause restenosis of the treated area (closure of an artery through cellular proliferation). Therefore, modulation of E2F activity allows control of cell proliferation and can be used to decrease proliferation and avoid closure of an artery. Examples of other such polynucleic acid decoys and target proteins include, but are not limited to, promoter sequences for inhibiting polymerases and ribosome binding sequences for inhibiting ribosomes. It is understood that the invention includes polynucleic acid decoys constructed to inhibit any target cellular factor.

A “gene therapy agent” refers to an agent that causes expression of a gene product in a target cell through introduction of a gene into the target cell followed by expression of the gene product. An example of such a gene therapy agent would be a genetic construct that causes expression of a protein, such as insulin, when introduced into a cell. Alternatively, a gene therapy agent can decrease expression of a gene in a target cell. An example of such a gene therapy agent would be the introduction of a polynucleic acid segment into a cell that would integrate into a target gene and disrupt expression of the gene. Examples of such agents include viruses and polynucleotides that are able to disrupt a gene through homologous recombination. Methods of introducing and disrupting genes within cells are well known to those of skill in the art.

An oligonucleotide of the invention can have any suitable length. Specifically, the oligonucleotide can be about 2 to about 100 nucleotides in length, inclusive; up to about 20 nucleotides in length, inclusive; or about 15 to about 30 nucleotides in length, inclusive. The oligonucleotide can be single-stranded or double-stranded. In one embodiment, the oligonucleotide can be single-stranded. The oligonucleotide can be DNA or RNA. In one embodiment, the oligonucleotide can be DNA. In one embodiment, the oligonucleotide can be synthesized according to commonly known chemical methods. In another embodiment, the oligonucleotide can be obtained from a commercial supplier. The oligonucleotide can include, but is not limited to, at least one nucleotide analog, such as bromo derivatives, azido derivatives, fluorescent derivatives or a combination thereof. Nucleotide analogs are well known to those of skill in the art. The oligonucleotide can include a chain terminator. The oligonucleotide can also be used, e.g., as a cross-linking reagent or a fluorescent tag. Many common linkages can be employed to couple an oligonucleotide to another moiety, e.g., phosphate, hydroxyl, etc. Additionally, a moiety may be linked to the oligonucleotide through a nucleotide analog incorporated into the oligonucleotide. In another embodiment, the oligonucleotide can include a phosphodiester linked 3′-5′ and 5′-3′ oligonucleotide backbone. Alternatively, the oligonucleotide can include non-phosphodiester linkages, such as phosphotioate type, phosphoramidate and peptide-nucleotide backbones. In another embodiment, moieties can be linked to the backbone sugars of the oligonucleotide. Methods of creating such linkages are well known to those of skill in the art.

Nucleotide and nucleoside analogues are well known on the art. Examples of such nucleoside analogs include, but are not limited to, Cytovene® (Roche Laboratories), Epivir® (Glaxo Wellcome), Gemzar® (Lilly), Hivid® (Roche Laboratories), Rebetron® (Schering), Videx® (Bristol-Myers Squibb), Zerit® (Bristol-Myers Squibb), and Zovirax® (Glaxo Wellcome). See, Physician's Desk Reference, 2005 Edition.

Polypeptides acting as additional bioactive agents dispersed within the polymers in the invention wound dressings, implants and coatings on other implantable surgical devices can have any suitable length. Specifically, the polypeptides can be about 2 to about 5,000 amino acids in length, inclusive; about 2 to about 2,000 amino acids in length, inclusive; about 2 to about 1,000 amino acids in length, inclusive; or about 2 to about 100 amino acids in length, inclusive.

The polypeptides can also include “peptide mimetics.” Peptide analogs are commonly used in the pharmaceutical industry as non-peptide bioactive agents with properties analogous to those of the template peptide. These types of non-peptide compound are termed “peptide mimetics” or “peptidomimetics.” Fauchere, J. (1986) Adv. Bioactive Agent Res., 15:29; Veber and Freidinger (1985) TINS p. 392; and Evans et al. (1987) J. Med. Chem., 30:1229; and are usually developed with the aid of computerized molecular modeling. Generally, peptidomimetics are structurally similar to a paradigm polypeptide (i.e., a polypeptide that has a biochemical property or pharmacological activity), but have one or more peptide linkages optionally replaced by a linkage selected from the group consisting of: —CH₂NH—, —CH₂S—, CH₂—CH₂—, —CH═CH—(cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends. Pharm. Sci., (1980) pp. 463-468 (general review); Hudson, D. et al., Int. J. Pept. Prot. Res., (1979) 14:177-185 (—CH₂ NH—, CH₂CH₂—); Spatola, A. F. et al., Life Sci., (1986) 38:1243-1249 (—CH₂—S—); Harm, M. M., J. Chem. Soc. Perkin Trans I (1982) 307-314 (—CH═CH—, cis and trans); Almquist, R. G. et al., J. Med. Chem., (1980) 23:2533 (—COCH₂—); Jennings-Whie, C. et al., Tetrahedron Lett., (1982) 23:2533 (—COCH₂—); Szelke, M. et al., European Appln., EP 45665 (1982) (—CH(OH)CH₂—); Holladay, M. W. et al., Tetrahedron Lett., (1983) 24:4401-4404 (—C(OH)CH₂—); and Hruby, V.J., Life Sci., (1982) 31:189-199 (—CH₂—S—). Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Additionally, substitution of one or more amino acids within a polypeptide with a D-Lysine in place of L-lysine) may be used to generate more stable polypeptides and polypeptides resistant to endogenous proteases.

In one embodiment, the additional bioactive agent polypeptide dispersed in the polymers or hydrogels used in the invention wound dressings, implants and coatings of surgical devices can be an antibody. In one embodiment, the antibody can bind to a cell adhesion molecule, such as a cadherin, integrin or selectin. In another embodiment, the antibody can bind to an extracellular matrix molecule, such as collagen, elastin, fibronectin or laminin. In still another embodiment, the antibody can bind to a receptor, such as an adrenergic receptor, B-cell receptor, complement receptor, cholinergic receptor, estrogen receptor, insulin receptor, low-density lipoprotein receptor, growth factor receptor or T-cell receptor. Antibodies attached to polymers (either directly or by a linker) can also bind to platelet aggregation factors (e.g., fibrinogen), cell proliferation factors (e.g., growth factors and cytokines), and blood clotting factors (e.g., fibrinogen). In another embodiment, an antibody can be conjugated to an active agent, such as a toxin. In another embodiment, the antibody can be Abciximab (ReoProR)). Abciximab is a Fab fragment of a chimeric antibody that binds to beta(3) integrins. Abciximab is specific for platelet glycoprotein IIb/IIIa receptors, e.g., on blood cells. Human aortic smooth muscle cells express alpha(v)beta(3) integrins on their surface. Treating beta(3) expressing smooth muscle cells may prohibit adhesion of other cells and decrease cellular migration or proliferation. Abciximab also inhibits aggregation of blood platelets.

Useful anti-platelet or anti-coagulation agents that may be used include, e.g., Coumadin® (DuPont), Fragmin® (Pharmacia & Upjohn), Heparin® (Wyeth-Ayerst), Lovenox®, Normiflo®, Orgaran® (Organon), Aggrastat® (Merck), Agrylin® (Roberts), Ecotrin® (Smithkline Beecham), Flolan® (Glaxo Wellcome), Halfprin® (Kramer), Integrillin® (COR Therapeutics), Integrillin® (Key), Persantine® (Boehringer Ingelheim), Plavix® (Bristol-Myers Squibb), ReoPro® (Centecor), Ticlid® (Roche), Abbokinase® (Abbott), Activase® (Genentech), Eminase® (Roberts), and Strepase® (Astra). See, Physician's Desk Reference, 2005 Edition. Specifically, the anti-platelet or anti-coagulation agent can include trapidil (avantrin), cilostazol, heparin, hirudin, or ilprost.

Trapidil is chemically designated as N,N-dimethyl-5-methyl-[1,2,4]triazolo[1,-5-a]pyrimidin-7-amine.

Cilostazol is chemically designated as 6-[4-(1-cyclohexyl-1H-tetrazol-5-yl)-butoxy]-3,4-dihydro-2(1H)-quinolinone.

Heparin is a glycosaminoglycan with anticoagulant activity; a heterogeneous mixture of variably sulfonated polysaccharide chains composed of repeating units of D-glucosamine and either L-iduronic or D-glucuronic acids.

Hirudin is an anticoagulant protein extracted from leeches, e.g., Hirudo medicinalis.

Iloprost is chemically designated as 5-[Hexahydro-5-hydroxy-4-(3-hydroxy-4-methyl-1-octen-6-ynyl)-2(1H)-pentalenylidene]pentanoic acid.

The immune suppressive agent can include, e.g., Azathioprine® (Roxane), BayRho-D® (Bayer Biological), CellCept® (Roche Laboratories), Imuran® (Glaxo Wellcome), MiCRhoGAM® (Ortho-Clinical Diagnostics), Neoran® (Novartis), Orthoclone OKT3® (Ortho Biotech), Prograf® (Fujisawa), PhoGAM® (Ortho-Clinical Diagnostics), Sandimmune® (Novartis), Simulect® (Novartis), and Zenapax® (Roche Laboratories).

Specifically, the immune suppressive agent can include rapamycin or thalidomide. Rapamycin is a triene macrolide isolated from Streptomyces hygroscopicus.

Thalidomide is chemically designated as 2-(2,6-dioxo-3-piperidinyl)-1H-iso-indole-1,3(2H)-dione.

Anti-cancer or anti-cell proliferation agents that can be incorporated as an additional bioactive agent in the invention wound dressings, implants and device coatings include, e.g., nucleotide and nucleoside analogs, such as 2-chloro-deoxyadenosine, adjunct antineoplastic agents, alkylating agents, nitrogen mustards, nitrosoureas, antibiotics, antimetabolites, hormonal agonists/antagonists, androgens, antiandrogens, antiestrogens, estrogen & nitrogen mustard combinations, gonadotropin releasing hormone (GNRH) analogues, progestrins, immunomodulators, miscellaneous antineoplastics, photosensitizing agents, and skin and mucous membrane agents. See, Physician's Desk Reference, 2005 Edition.

Suitable adjunct antineoplastic agents include Anzemet® (Hoeschst Marion Roussel), Aredia® (Novartis), Didronel® (MGI), Diflucan® (Pfizer), Epogen® (Amgen), Ergamisol® (Janssen), Ethyol® (Alza), Kytril® (SmithKline Beecham), Leucovorin® (Immunex), Leucovorin® (Glaxo Wellcome), Leucovorin® (Astra), Leukine® (Immunex), Marinol® (Roxane), Mesnex® (Bristol-Myers Squibb Oncology/Immunology), Neupogen (Amgen), Procrit® (Ortho Biotech), Salagen® (MGI), Sandostatin® (Novartis), Zinecard® (Pharmacia and Upjohn), Zofran® (Glaxo Wellcome) and Zyloprim® (Glaxo Wellcome).

Suitable miscellaneous alkylating agents include Myleran® (Glaxo Wellcome), Paraplatin® (Bristol-Myers Squibb Oncology/Immunology), Platinol® (Bristol-Myers Squibb Oncology/Immunology) and Thioplex® (Immunex).

Suitable nitrogen mustards include Alkeran® (Glaxo Wellcome), Cytoxan® (Bristol-Myers Squibb Oncology/Immunology), Ifex® (Bristol-Myers Squibb Oncology/Immunology), Leukeran® (Glaxo Wellcome) and Mustargen® (Merck).

Suitable nitrosoureas include BiCNU® (Bristol-Myers Squibb Oncology/Immunology), CeeNU® (Bristol-Myers Squibb Oncology/Immunology), Gliadel® (Rhone-Poulenc Rover) and Zanosar® (Pharmacia and Upjohn).

Suitable antimetabolites include Cytostar-U® (Pharmacia and Upjohn), Fludara® (Berlex), Sterile FUDR® (Roche Laboratories), Leustatin® (Ortho Biotech), Methotrexate® (Immunex), Parinethol® (Glaxo Wellcome), Thioguanine® (Glaxo Wellcome) and Xeloda® (Roche Laboratories).

Suitable androgens include Nilandron® (Hoechst Marion Roussel) and Teslac®(Bristol-Myers Squibb Oncology/Immunology).

Suitable antiandrogens include Casodex® (Zeneca) and Eulexin® (Schering).

Suitable antiestrogens include Arimidex® (Zeneca), Fareston® (Schering), Femara® (Novartis) and Nolvadex® (Zeneca).

Suitable estrogen and nitrogen mustard combinations include Emcyt® (Pharmacia and (Upjohn).

Suitable estrogens include Estrace® (Bristol-Myers Squibb) and Estrab® (Solvay).

Suitable gonadotropin releasing hormone (GNRH) analogues include Leupron Depot® (TAP) and Zoladex® (Zeneca).

Suitable progestins include Depo-Provera® (Pharmacia and Upjohn) and Megace®(Bristol-Myers Squibb Oncology/Immunology).

Suitable immunomodulators include Erganisol® (Janssen) and Proleukin® (Chiron (Corporation).

Suitable miscellaneous antineoplastics include Camptosar® (Pharmacia and Upjohn), Celestone® (Schering), DTIC-Dome® (Bayer), Elspar® (Merck), Etopophos® (Bristol-Myers Squibb Oncology/Immunology), Etopoxide® (Astra), Gemzar® (Lilly), Hexalen® (U.S. Bioscience), Hycantin® (SmithKline Beecham), Hydrea® (Bristol-Myers Squibb Oncology/Immunology), Hydroxyurea® (Roxane), Intron A® (Schering), Lysodren® (Bristol-Myers Squibb Oncology/Immunology), Navelbine® (Glaxo Wellcome), Oncaspar® (Rhone-Poulenc Rover), Oncovin® (Lilly), Proleukin® (Chiron Corporation), Rituxan® (IDEC), Rituxan® (Genentech), Roferon-A® (Roche Laboratories), Taxol® (paclitaxol/paclitaxel, Bristol-Myers Squibb Oncology/Immunology), Taxotere® (Rhone-Poulenc Rover), TheraCys® (pasteur Merieux Connaught), Tice BCG® (Organon), Velban® (Lilly), VePesid® (Bristol-Myers Squibb Oncology/Immunology), Vesanoid® (Roche Laboratories) and Vumon® (Bristol-Myers Squibb Oncology/Immunology).

Suitable photosensitizing agents include Photofrin® (Sanofi).

Specifically, useful anti-cancer or anti-cell proliferation agents can include Taxol® (paclitaxol), a nitric oxide-releasing compound, or NicOX (NCX-4016). Taxol® (paclitaxol) is chemically designated as 5β,20-Epoxy-1,2α4,7β,10β,13α-hexahydroxytax-11-en-9-one 4,10-diacetate 2-benzoate 13-ester with (2R,3S)-N-benzoyl-3-phenylisoserine. NCX-4016 is chemically designated as 2-acetoxy-benzoate 2-(nitroxymethyl)-phenyl ester, and is an antithrombotic agent.

Preferred wound healing agents for dispersion into and release from the biodegradable polymers used in the invention wound healing compositions, such as wound dressings, implants and surgical device coatings, include anti-proliferants, such as rapamycin and any of its analogs or derivatives, paclitaxel or any of its taxene analogs or derivatives, everolimus, Sirolimus (a potent inhibitor of the growth of smooth muscle cells in blood vessels), Everolimus (an immunosuppressant that blocks growth factor-mediated proliferation of hematopoietic and non-hematopoietic cells), tacrolimus (used, e.g., to prevent liver transplant rejection, in Cohn's Disease and ulcerative colitis and as treatment for atomic eczema), or any of its—limus named family of drugs. Also preferred are members of the stating family, such as simvastatin, atorvastatin, fluvastatin, pravastatin, lovastatin, rosuvastatin, geldanamycins, such as 17AAG (17-allylamino-17-demethoxygeldanamycin); Epothilone D and other epothilones, 17-dimethylaminoethylamino-17-demethoxy-geldanamycin and other polyketide inhibitors of heat shock protein 90 (Hsp90), Cilostazol, and the like. Such anti-proliferant bioactive agents, for example, may be dispersed in a sheet of PEA or PEUR polymers and used as a surgical wrap. For example, such a surgical wrap can be applied to the exterior of an anastomosis, a site of stent implant, or arterio-venous graft or fistula to reduce restenosis and development of scar tissue.

It is appreciated that those skilled in the art understand that the bioactive agent useful in the present invention is the bioactive substance present in any of the bioactive agents or additional bioactive agents disclosed above. For example, Taxol® is typically available as an injectable, slightly yellow viscous solution. The bioactive agent, however, is a crystalline powder with the chemical name 5β,20-Epoxy-1,2α,4,7β,10β,13α-hexahydroxytax-11-en-9-one 4,10-diacetate 2-benzoate 13-ester with (2R,3S)-N-benzoyl-3-phenylisoserine. Physician's Desk Reference (PDR), Medical Economics Company (Montvale, N.J.), (53rd Ed.), pp. 1059-1067.

As used herein a “residue of a bioactive agent” or “residue of an additional bioactive agent” is a radical of such bioactive agent as disclosed herein having one or more open valences. Any synthetically feasible atom or atoms of the bioactive agent can be removed to provide the open valence, provided bioactivity is substantially retained when the radical is attached to a residue of compound (I)-(VI). Based on the linkage that is desired, those skilled in the art can select suitably functionalized starting materials that can be derived from a bioactive agent using procedures that are known in the art.

The residue of a bioactive agent can be formed employing any suitable reagents and reaction conditions. Suitable reagents and reaction conditions are disclosed, e.g., in Advanced Organic Chemistry, Part B: Reactions and Synthesis, Second Edition, Carey and Sundberg (1983); Advanced Organic Chemistry, Reactions, Mechanisms and Structure, Second Edition, March (1977); and Comprehensive Organic Transformations, Second Edition, Larock (1999).

In certain embodiments, the polymer/bioactive agent linkage degrades to provide a suitable and effective amount of free bioactive agent. As will be appreciated by those of skill in the art, depending upon the chemical and therapeutic properties of the biological agent, in certain other embodiments, the bioactive agent attached to the polymer performs its therapeutic effect while still attached to the polymer, such as is the case with the “sticky” polypeptides Protein A and Protein G, known herein as “ligands”, which function while attached to the polymer to hold a target molecule close to the polymer, and the bradykinins and antibodies, which function by contacting (e.g., bumping into) a receptor on a target molecule. Any suitable and effective amount of bioactive agent can be released from the wound dressing and will typically depend, e.g., on the specific polymer, type of bioactive agent, and the particular mode of dispersion, for example the type polymer/bioactive agent linkage chosen. Typically, up to about 100% of the bioactive agent can be released from the polymer by degradation of the polymer. Specifically, up to about 90%, up to 75%, up to 50%, or up to 25% of the bioactive agent can be released from the polymer. Factors that typically affect the amount of the bioactive agent that is released from the polymer are the type of polymer/bioactive agent linkage, and the nature and amount of additional substances present in the composition.

The polymer/bioactive agent linkage can be selected to degrade over a desired period of time to provide time release of a suitable and effective amount of bioactive agent according to the type of wound being treated. Any suitable and effective period of time can be chosen by judicious choice of the chemical properties of the linkage of the bioactive agent to the polymer. Typically, the suitable and effective amount of bioactive agent can be released over a time selected from about twenty-four hours, about seven days, about thirty days, about ninety days, and about one hundred and twenty days. Longer time spans are particularly suitable for implantable wound dressings and device coatings. Additional factors that typically affect the length of time over which the bioactive agent is released from the polymer include, e.g., the nature and amount of polymer, the nature and amount of bioactive agent, and the nature and amount of additional substances present in the composition.

Polymer/Linker/Bioactive Agent Linkage

In addition to being directly linked to the residue of a compound of formula (I)-(VI), the residue of a bioactive agent can also be linked to the residue of a compound of formula (I)-(VI) by a suitable linker. The structure of the linker is not crucial, provided the resulting compound of the invention has an effective therapeutic index as a bioactive agent.

Suitable linkers include linkers that separate the residue of a compound of formula (I)-(VI) from the residue of a bioactive agent by a distance of about 5 angstroms to about 200 angstroms, inclusive. Other suitable linkers include linkers that separate the residue of a compound of formula (I)-(VI) and the residue of a bioactive agent by a distance of about 5 angstroms to about 100 angstroms, inclusive, as well as linkers that separate the residue of a compound of formula (I)-(VI) from the residue of a bioactive agent by a distance of about 5 angstroms to about 50 angstroms, or by about 5 angstroms to about 25 angstroms, inclusive.

The linker can be linked to any synthetically feasible position on the residue of a compound of formula (I)-(VI). Based on the linkage that is desired, those skilled in the art can select suitably functionalized starting materials that can be derived from a compound of formula (I)-(VI) and a bioactive agent using procedures that are known in the art.

The linker can conveniently be linked to the residue of a compound of formula (I)-(VI) or to the residue of a bioactive agent through an amide (e.g., —N(R)C(═O)— or —C(═O)N(R)—), ester (e.g., —OC(═O)— or —C(—O)O—), ether (e.g., —O—), ketone (e.g., —C(═O)—) thioether (e.g., —S—), sulfinyl (e.g., —S(O)—), sulfonyl (e.g., —S(O)₂—), disulfide (e.g., —S—S—), amino (e.g., —N(R)—) or a direct (e.g., C—C) linkage, wherein each R is independently H or (C₁-C₆)alkyl. The linkage can be formed from suitably functionalized starting materials using synthetic procedures that are known in the art. Based on the linkage that is desired, those skilled in the art can select suitably functionalized starting materials that can be derived from a residue of a compound of formula (I)-(VI), a residue of a bioactive agent, and from a given linker using procedures that are known in the art.

Specifically, the linker can be a divalent radical of the formula W-A-Q wherein A is (C₁-C₂₄)alkyl, (C₂-C₂₄)alkenyl, (C₂-C₂₄)alkynyl, (C₃-C₈)cycloalkyl, or (C₆-C₁₀)aryl, wherein W and Q are each independently —N(R)C(═O)—, —C(═O)N(R)—, OC(═O)—, —C(═O)O—, —O—, —S—, —S(O)—, —S(O)₂—, —S—S—, —N(R)—, —C(═O)—, or a direct bond (i.e., W and/or Q is absent); wherein each R is independently H or (C₁-C₆)alkyl.

Specifically, the linker can be a divalent radical of the formula W—(CH₂)ₙ-Q, wherein n is from about 1 to about 20, from about 1 to about 15, from about 2 to about 10, from about 2 to about 6, or from about 4 to about 6; wherein W and Q are each independently —N(R)C(═O)—, —C(═O)N(R)—, —OC(═O)—, —C(═O)O—, —O—, —S—, —S(O)—, —S(O)₂—, —S—S—, —C(═O)—, —N(R)—, or a direct bond (i.e., W and/or Q is absent); wherein each R is independently H or (C₁-C₆)alkyl.

Specifically, W and Q can each independently be —N(R)C(═O)—, —C(═O)N(R)—, —OC(═O)—, —N(R)—, —C(═O)O—, —O—, or a direct bond (i.e., W and/or Q is absent).

Specifically, the linker can be a divalent radical formed from a saccharide.

Specifically, the linker can be a divalent radical formed from a cyclodextrin.

Specifically, the linker can be a divalent radical, i.e., divalent radicals formed from a peptide or an amino acid. The peptide can comprise 2 to about 25 amino acids, 2 to about 15 amino acids, or 2 to about 12 amino acids.

Specifically, the peptide can be poly-L-lysine (i.e., [—NHCH[(CH₂)₄NH₂]CO—]ₘ-Q wherein Q is H, (C₁-C₁₄)alkyl, or a suitable carboxy protecting group; and wherein m is about 2 to about 25. The poly-L-lysine can contain about 5 to about 15 residues (i.e., m is from about 5 to about 15). For example, the poly-L-lysine can contain from about 8 to about 11 residues (i.e., m is from about 8 to about 11).

Specifically, the peptide can also be poly-L-glutamic acid, poly-L-aspartic acid, poly-L-histidine, poly-L-ornithine, poly-L-serine, poly-L-threonine, poly-L-tyrosine, poly-L-leucine, poly-L-lysine-L-phenylalanine, poly-L-arginine, or poly-L-lysine-L-tyrosine.

Specifically, the linker can be prepared from 1,6-diaminohexane H₂N(CH₂)₆NH₂, 1,5-diaminopentane H₂N(CH₂)₅NH₂, 1,4-diaminobutane H₂N(CH₂)₄NH₂, or 1,3-diaminopropane H₂N(CH₂)₃NH₂.

One or more bioactive agents can be linked to the polymer through a linker. Specifically, the residue of each of the bioactive agents can each be linked to the residue of the polymer through a linker. Any suitable number of bioactive agents (i.e., residues thereof) can be linked to the polymer (i.e., residue thereof) through a linker. The number of bioactive agents that can be linked to the polymer through a linker can typically depend upon the molecular weight of the polymer. For example, for a compound of formula (VI), wherein n is about 50 to about 150, up to about 450 bioactive agents (i.e., residues thereof) can be linked to the polymer (i.e., residue thereof) through a linker, up to about 300 bioactive agents (i.e., residues thereof) can be linked to the polymer (i.e., residue thereof) through a linker, or up to about 150 bioactive agents (i.e., residues thereof) can be linked to the polymer (i.e., residue thereof) through a linker. Likewise, for a compound of formula (II), wherein n is about 50 to about 150, up to about 10 to about 450 bioactive agents (i.e., residues thereof) can be linked to the polymer (i.e., residue thereof) through a linker, up to about 300 bioactive agents (i.e., residues thereof) can be linked to the polymer (i.e., residue thereof) through a linker, or up to about 150 bioactive agents (i.e., residues thereof) can be linked to the polymer (i.e., residue thereof) through a linker.

In one embodiment of the present invention, a polymer (i.e., residue thereof) as disclosed herein can be linked to the linker via a carboxyl group (e.g., COOR²) of the polymer.

For example, a compound of formula (II), wherein R² is independently hydrogen, or (C₆-C₁₀)aryl(C₁-C₆)alkyl, can react with an amino functional group of the linker or a hydroxyl functional group of the linker, to provide a Polymer/Linker having an amide linkage or a Polymer/Linker having a carboxyl ester linkage, respectively. In another embodiment, the carboxyl group can be transformed into an acyl halide or an acyl anhydride.

In one embodiment of the invention, a bioactive agent (i.e., residue thereof) can be linked to the linker via a carboxyl group (e.g., COOR, wherein R is hydrogen, (C₆-C₁₀)aryl(C₁-C₆)alkyl or (C₁-C₆)alkyl) of the linker. Specifically, an amino functional group of the bioactive agent or a hydroxyl functional group of the bioactive agent can react with the carboxyl group of the linker, to provide a Linker/Bioactive agent having an amide linkage or a Linker/Bioactive agent having a carboxylic ester linkage, respectively. In another embodiment, the carboxyl group of the linker can be transformed into an acyl halide or an acyl anhydride.

The polymer/linker/bioactive agent linkage can degrade to provide a suitable and effective amount of bioactive agent. Any suitable and effective amount of bioactive agent can be released and will typically depend, e.g., on the specific polymer, bioactive agent, linker, and polymer/linker/bioactive agent linkage chosen. Typically, up to about 100% of the bioactive agent can be released from the polymer/linker/bioactive agent. Specifically, up to about 90%, up to 75%, up to 50%, or up to 25% of the bioactive agent can be released from the polymer/linker/bioactive agent. Factors that typically affect the amount of the bioactive agent released from the polymer with linked bioactive agent include, e.g., the nature and amount of polymer, the nature and amount of bioactive agent, the nature and amount of linker, the nature of the polymer/linker/bioactive agent linkage, and the nature and amount of additional substances present in the composition.

The polymer/linker/bioactive agent linkage can degrade over a period of time to provide the suitable and effective amount of bioactive agent. Any suitable and effective period of time can be chosen. Typically, the suitable and effective amount of bioactive agent can be released in about twenty-four hours, in about seven days, in about thirty days, in about ninety days, or in about one hundred and twenty days. Factors that typically affect the length of time in which the bioactive agent is released from the polymer/linker/bioactive agent include, e.g., the nature and amount of polymer, the nature and amount of bioactive agent, the nature of the linker, the nature of the polymer/linker/bioactive agent linkage, and the nature and amount of additional substances present in the composition.

Polymer Intermixed with Wound Healing Agent or Additional Bioactive Agent

In addition to being linked to one or more wound healing agents, either directly or through a linker, a polymer in the invention wound healing composition described herein can be physically intermixed with one or more wound healing agents or additional bioactive agents to provide the invention composition.

As used herein, “intermixed” refers to a polymer as described herein physically mixed with a bioactive agent, or a polymer as described herein that is physically in contact with a bioactive agent. The composition so formed may have one or more bioactive agents present on the surface of the polymer, partially embedded in the polymer, or completely embedded in the polymer. Additionally, the composition may include a polymer as described herein and a bioactive agent in a homogeneous composition (i.e., a homogeneous composition).

Any suitable amount of polymer and bioactive agent can be employed to provide the composition. The polymer can be present in about 0.1 wt. % to about 99.9 wt. % of the composition. Typically, the polymer can be present above about 25 wt. % of the composition; above about 50 wt. % of the composition; above about 75 wt. % % of the composition; or above about 90 wt. % of the composition. Likewise, the bioactive agent can be present in about 0.1 wt. % to about 99.9 wt. % of the composition. Typically, the bioactive agent can be present above about 5 wt. % of the composition; above about 10 wt. % of the composition; above about 15 wt. % of the composition; or above about 20 wt. % of the composition.

In yet another embodiment of the invention the polymer/bioactive agent, polymer/linker/bioactive agent, composition, or combination thereof as described herein, can be applied, as a polymeric film onto at least a portion of the surface of a surgical device (e.g., stent structure). The surface of the surgical device can be coated with the polymeric film. The polymeric film can have any suitable thickness on the surgical device. For example, the thickness of the polymeric film on the surgical device can be about 1 to about 50 microns thick or about 5 to about 20 microns thick. In the invention stents and multilayered stents, each of the layers can be from 0.1 micron to 50 microns thick, for example from 0.5 micron to 5 microns in thickness.

The polymeric film can effectively serve as a bioactive agent-eluting polymeric coating on a surgical device, such as a stent structure, orthopedic implant, and the like. This bioactive agent-eluting polymeric coating can be created on the surgical device by any suitable coating process, e.g., dip coating, vacuum depositing, or spray coating the polymeric film, on the surgical device to create a type of local bioactive agent delivery system.

The wound healing agent-eluting polymer can be used in conjunction with, e.g., hydrogel-based bioactive agent delivery systems. For example, in one embodiment, the composition is used in a multilayered wound dressing wherein the above-described polymer is in the form of a sheet or pad of woven or amorphous fibers. At least one surface of the sheet or pad is optionally coated with an additional composition layer in a sandwich type of configuration to deliver to the blood capillaries wound healing agents that promote natural re-endothelialization processes. Such an additional composition layer may comprise a hydrogel, as described herein, that comprises at least one bioactive agent or additional bioactive agent dispersed in the hydrogel. The hydrogel layer optionally may provide an elution rate different than that of the polymer sheet or pad of the wound dressing. Optionally the multilayered wound dressing may further include an occlusive layer, e.g., to be placed externally to the wound, to substantially prevent fluid penetration, either liquid or gas, through the wound dressing.

Any suitable size of polymer and bioactive agent can be employed to provide the invention wound healing compositions. For example, the polymer can have a size of less than about 1×10⁻⁴ meters, less than about 1×10⁻⁵ meters, less than about 1×10⁻⁶ meters, less than about 1×10⁻⁷ meters, less than about 1×10⁻⁸ meters, or less than about 1×10⁻⁹ meters.

The composition can degrade to release a suitable and effective amount of the wound healing agent and optional additional bioactive agent. Any suitable and effective amount of such bioactive agents can be released and will typically depend, e.g., on the specific composition chosen. Typically, up to about 100% of the bioactive agent(s) can be released from the composition. Specifically, up to about 90%, up to 75%, up to 50%, or up to 25% of the bioactive agent(s) can be released from the composition. Factors that typically affect the amount of the bioactive agent that is released from the composition include, e.g., the nature and amount of polymer, the nature and amount of bioactive agent, and the nature and amount of additional substances present in the composition.

The composition can degrade over a period of time to provide the suitable and effective amount of bioactive agent. Any suitable and effective period of time can be chosen. For example, the polymer can be selected to release the bioactive agent over about twenty-four hours, over about two days, over about seven days, over about ninety days, or over about one hundred and twenty days, the latter being particularly useful when an implantable wound dressing is desired. Factors that typically affect the length of time over which the bioactive agent is released from the composition include, e.g., the nature and amount of polymer, the nature and amount of bioactive agent, and the nature and amount of additional substances present in the composition.

In another embodiment, the invention provides an invention composition (e.g., for use in a wound dressing) that includes a polymer as described herein physically intermixed with one or more bioactive agents. The polymer that is present in the composition can also be linked, either directly or through a linker, to one or more (e.g., 1, 2, 3, or 4) bioactive agents. As such, the polymer can be intermixed with one or more (e.g., 1, 2, 3, or 4) bioactive agents and can be linked, either directly or through a linker, to one or more (e.g., 1, 2, 3, or 4) bioactive agents.

In one embodiment, the polymer is physically intermixed with at least one bioactive agent. In another embodiment, the polymer is linked to at least one bioactive agent, either directly or through a linker. In another embodiment, the polymer is linked to one or more bioactive agents, either directly or through a linker, and the resulting polymer can also be physically intermixed with one or more bioactive agents.

In yet another embodiment, the invention provides methods for promoting natural healing of a wound comprising contacting the wound with an invention wound dressing under conditions suitable for promoting natural healing of the wound. The natural healing process includes re-endothelialization of the wound bed (e.g., closure of the wound).

To this end, in treating a chronic wound, the polymer of the wound dressing can be placed in contact with the wound bed and the polymer can be allowed to biodegrade, releasing the bioactive agent into the wound bed while the polymer is absorbed therein. Alternatively, the wound dressing used in treatment of a chronic wound will include a biodegradable hydrogel layer (i.e., non-stick layer), which can be placed in contact with the wound bed. The hydrogel is allowed to biodegrade, releasing the bioactive agent into the wound bed. The compositions of the polymer layer and the hydrogel layer can be selected to release their respective bioactive agents at different rates. The invention methods are beneficially used in treatment of such chronic wounds as venous stasis ulcer, diabetic ulcer, pressure ulcer, or ischemic ulcer.

The invention will be further understood with reference to the following examples, which are purely exemplary, and should not be taken as limiting the true scope of the present invention as described in the claims.

### EXAMPLES

### Example 1

Amide Bond Formation This example illustrates the coupling of a carboxyl group of a polymer with an amino functional group of the bioactive agent, or equally, the coupling of a carboxyl group of the bioactive agent with an amino functional group of a polymer.

Coupling Through Pre-Formed Active Esters; Carbodiimide Mediated Couplings—Conjugation of 4-Amino-Tempo to Polymer The free carboxylic acid form of the PEA polymer is converted first to its active succinimidyl ester (PEA-OSu) or benzotriazolyl ester (PEA-OBt). This conversion can be achieved by reacting dried PEA-H polymer (i.e. PEA with free pendant carboxylic acids) with N-Hydroxysuccinimide (NHS) or 1-Hydroxybenzotriazole (HOBt) and a suitable coupling agent, such as dicyclohexylcarbodiimide (DCC), in anhydrous CH₂Cl₂ at room temperature for 16 hrs. After filtering away the precipitated dicyclohexylurea (DCU), the PEA-OSu product may be isolated by precipitation, or used without further purification, in which case the PEA-OSu solution is transferred to a round bottom flask, diluted to the desired concentration, and cooled to 0° C. Next, a solution of the free amine-containing bioactive agent is added in a single shot at 0° C. The nucleophile of 4-amino TEMPO, specifically is the free amine substituted at position 4. Equally, the nucleophile of a bioactive agent may be revealed in situ by treating the ammonium salt of such a bioactive agent with a hindered base, preferably a tertiary amine, such as triethylamine or, diisopropylethylamine, in a suitable aprotic solvent, such as dichloromethane (DCM). Tracking consumption of the free amine by TLC, as indicated by ninhydrin staining, monitors the reaction. Work-up for the polymer involves customary precipitation of the reaction solution into a mixture of non-solvent, such as hexane/ethyl acetate. Solvent is then decanted, polymer residue is resuspended in a suitable solvent, filtered, concentrated by roto-evaporation, cast onto a clean teflon tray, and dried under vacuum to furnish the PEA-bioactive agent conjugate, specifically, PEA-4-Amino-Tempo.

Aminium/Uronium Salt and Phosphonium Salt Mediated Couplings. Two effective catalysts for this type of coupling include: HBTU, O-(benzotriazol-1-yl)-1,1,3,3-tetramethyluronium hexafluorophosphate, and BOP, 1-benzotriazolyloxytris(dimethylamino)phosphonium hexafluorophosphate (Castro's Reagent). These reagents are employed in the presence of equimolar amounts of the carboxyl group of the polymer and the amino functional group of the bioactive agent (neutral or as the ammonium salt), with a tertiary amine such as diisopropylethylamine, N-methylmorpholine, or dimethylamino-substituted pyridines (DMAP), in solvents such as DMF, THF, or DCM.

### Example 2

Ester Bond Formation This example illustrates coupling of a carboxyl group of a polymer with a hydroxyl functional group of the bioactive agent, or equally, coupling of a carboxyl group of the bioactive agent with a hydroxyl functional group of a polymer.

Carbodiimide Mediated Esterification For the conjugation, a sample of the carboxyl-group-containing polymer was dissolved in DCM. To this slightly viscous solution was added a solution of the hydroxyl-containing-drug/biologic and DMAP in DCM. The flask was then placed in an ice bath and cooled to 0° C. Next, a solution of 1,3-diisopropylcarbodiimide (DIPC) in DCM was added, the ice bath removed, and the reaction warmed to room temperature. The conjugation reaction was stirred at room temperature for 16 hours during which time TLC was periodically performed to monitor consumption of the hydroxyl functional group of the bioactive agent. After the allotted time, the reaction mixture was precipitated, and the Polymer-bioactive agent conjugate isolated as described above in Example 1.

### Example 3

This Example illustrates the effect of different concentrations of bioactive agents on adhesion and proliferation of epithelial cells (EC) and smooth muscle cells (SMC) on gelatin coated surfaces.

Human Coronary artery endothelial cells (EC) plated on gelatin coated culture plates were co-cultured with EC special media containing one of the bioactive agents shown in Table 1 below in the various concentrations shown.

TABLE 1

| | Bioagents | 100 μM | 10 μM | 1 μM | 100 nm |
|---|---|---|---|---|---|
| A | Bradykinin[Hyp 3] | 372 | 37.23 | 3.72 | 0.372 |
| B | Bradykinin | 322.8 | 32.28 | 3.228 | 0.3228 |
| C | Adenosine | 80.16 | 8.016 | 0.816 | 0.0816 |
| D | Sphingosine 1- | 113.85 | 11.385 | 1.1385 | 0.11385 |
| | Phosphate (S1P) | | | | |
| E | Lysophosphatidic | 137.55 | 13.755 | 1.375 | 0.1376 |
| | Acid (LPA) | | | | |
| F | Control | No | | | |
| | | additives | | | |

Cells cultured under similar conditions without adding bioagents are considered as ‘Control.’

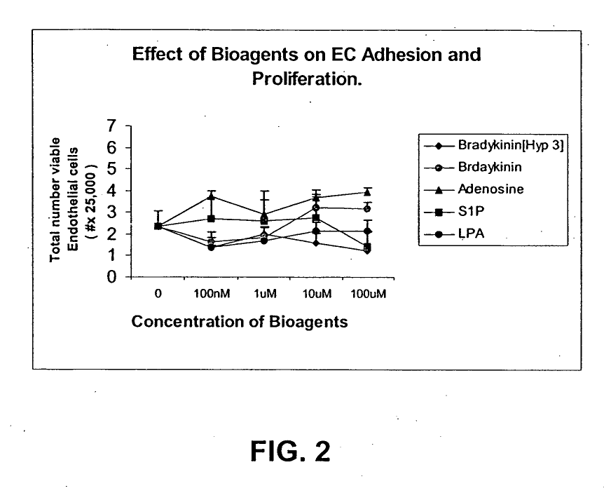

Twenty-four hours later the cells were observed microscopically, stained with trypan blue and counted. The results of the microscopic observation of cell morphology and confluency of culturing the EC in the presence of the Bioagents tested are summarized in Table 2 below. The effect of the various bioagents on EC adhesion and proliferation is shown graphically in FIG. 2.

TABLE 2
Microscopic observation for the EC morphology and Confluency in the presence of
Bioagents

| Bioagents | 100 nm | 1 μM | 10 μM | 100 μM |
|---|---|---|---|---|
| radykinin | Normal Cell | Normal Cell | Normal Cell | Normal Cell |
| [Hyp 3] | Morphology and | Morphology and | Morphology and | Morphology and |
| | proliferation. | proliferation. | proliferation. | proliferation. |
| | Less confluent than | Less confluent | Less confluent | Less confluent than |
| | control | than control | than control | control |
| Bradykinin | Normal Cell | Normal Cell | Normal Cell | Normal Cell |
| | Morphology and | Morphology and | Morphology and | Morphology and |
| | proliferation. | proliferation. | proliferation. | proliferation. |
| | More confluent than | More confluent | Less confluent | Less confluent than |
| | control | than control | than control | control |
| Adenosine | Normal Cell | Normal Cell | Normal Cell | Normal Cell |
| | Morphology and | Morphology and | Morphology and | Morphology and |
| | proliferation. | proliferation. | proliferation. | proliferation. |
| | More confluent than | More confluent | More confluent | More confluent than |
| | control | than control | than control | control |
| S1P | ˜70% of cells | ˜50% of cells | 25% of cells | 95% of cells exhibited |
| | adhered with normal | adhered with | adhered with | distorted morphology. |
| | morphology and | normal | normal | No proliferating cells |
| | proliferation | morphology and | morphology and | Aggregates of dead |
| | | Proliferation | proliferation. | cells were floating |
| | | | Lot of dead cells | |
| | | | were floating | |
| LPA | 70% cells Adhered | 50% cells | 30% cells | 10% cells Adhered and |
| | and exhibited | Adhered and | Adhered and | exhibited normal |
| | normal morphology. | exhibited normal | exhibited normal | morphology. |
| | Dead cells were | morphology. | morphology. | Big aggregates of dead |
| | floating | Dead cells were | Aggregates of | cells were floating |
| | | floating | dead cells were | |
| | | | floating | |
| Control | Normal | Normal | Normal | Normal Morphology, |
| | Morphology, and >85% | Morphology, and | Morphology, and | and >85% confluent |
| | confluent | >85% confluent | >85% confluent | |

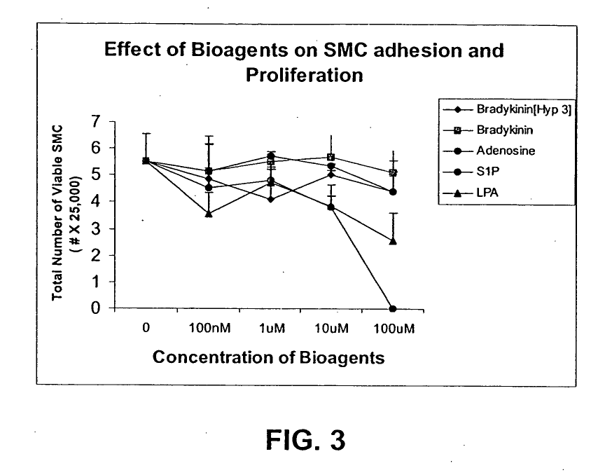

Effect of different concentrations of the bioagents listed above in Table 1 was also tested using human aortic smooth muscle cells (SMC) under similar conditions as described for EC. The results of the bioagents on adhesion and proliferation of SMC plated on gelatin coated culture plates are summarized in Table 3 below and shown graphically in FIG. 3.

TABLE 3
Microscopic observation for the SMC morphology and Confluency in the
presence of Bioagents

| Bioagents | 100 nm | 1 μM | 10 μM | 100 Mm |
|---|---|---|---|---|
| radykinin | Normal Cell | Normal Cell | Normal Cell | Normal Cell |
| [Hyp 3] | Morphology | Morphology | Morphology | Morphology |
| | and | and | and | and |
| | proliferation. | proliferation. | proliferation. | proliferation. |
| Bradykinin | Normal Cell | Normal Cell | Normal Cell | Distorted Cell |
| | Morphology | Morphology | Morphology | Morphology |
| | and | and | and | |
| | proliferation. | proliferation. | proliferation. | |
| | >70% | 70% | 50% | |
| | confluent | confluent | confluent | |
| Adenosine | Normal Cell | Distorted | 50% | >50% distorted |
| | Morphology | Cell | distorted | Cell |
| | and | Morphology | Cell | Morphology |
| | proliferation. | | Morphology | |
| S1P | Normal | ˜50% cell | 70% cells | 100% cells |
| | morphology. | adhered with | survived | died |
| | | normal | with | |
| | | morphology | distorted | |
| | | | morphology | |
| LPA | 70% cells | 50% cells | <50% cells | <10% cells |
| | Adhered and | Adhered and | Adhered and | Adhered and |
| | exhibited | exhibited | exhibited | exhibited |
| | normal | normal | normal | normal |
| | morphology. | morphology. | morphology. | morphology. |
| | | Lot of | | Big aggregates |
| | | dead cells | | of dead cells |
| | | were | | were floating |
| | | floating | | |
| Control | Normal | Normal | Normal | Normal |
| | Morphology, | Morphology, | Morphology, | Morphology, |
| | and >85% | and >85% | and >85% | and >85% |
| | confluent | confluent | confluent | confluent |

### Example 4

This Example reports a pre-clinical animal model evaluation of the Blue Medical coronary stent stainless steel stent structure (Blue Medical Devices, BV, Helmund, the Netherlands) coated with TEMPO polymer, in three stages: 1) Evaluation of post-implantation injury and inflammatory response, 2) Evaluation of in-stent neointimal hyperplasia, and 3) Comparison of TEMPO coated stents with the uncoated stents.

Stent Implantation Domestic crossbred pigs of both sexes weighing 20-25 kg were used for the study. The pigs were fed with a standard natural grain diet without lipid or cholesterol supplementation throughout the study. All animals were treated and cared for in accordance with the Belgium National Institute of Health Guide for the care and use of laboratory animals.

Acute Study In the acute study 2 uncoated stents and 2 each of 5 types of coated stents with differently dosed coatings (0% TEMPO Gamma, 50% TEMPO Gamma, 0% TEMPO ETO, 50% TEMPO ETO, 100% TEMPO+Top Layer ETO) were randomly implanted in the coronary arteries of 6 pigs. Pigs were sacrificed after 5 days to evaluate acute inflammatory response and thrombus formation caused by implantation of the stents. TEMPO=stent coated with 4-amino Tempo conjugated to the polymer; (Gamma=stent sterilized with gamma radiation; and ETO=stent sterilized with ethylene oxide.

Chronic Study In this study 8 uncoated stents and 8 TEMPO coated stents, 4 with 50% TEMPO and 4 with 100% TEMPO, were randomly implanted in the coronary arteries of selected pigs. The pigs were sacrificed after 6 weeks to evaluate peri-strut inflammation and neointimal hyperplasia. Surgical procedure and stent implantation in the coronary arteries were performed according to the methods described by De Scheerder et al. (Atherosclerosis. (1995) 114:105-114 and Coron Artery Dis. (1996) 7:161-166.

Prior to stent implantation, a balloon catheter was used as a reference to expand the stents to obtain an over-sizing of the artery of 10% to 20%, thereby causing damage to endothelium.

Quantitative Coronary Angiography Angiographic analysis of stented vessel segments was performed before stenting, immediately after, and at follow-up using the Polytron 1000®-system as described previously by De Scheerder et al. The diameter of the vessel segments was measured before and immediately after stent implantation, and at follow-up 6 weeks after implantation. The degree of over-sizing was expressed as measured maximum balloon size minus selected artery diameter divided by selected artery diameter.

Histopathology and Morphometry Coronary segments were carefully dissected, leaving a 1 cm minimum vessel length attached both proximal and distal to the stent. The segments were fixed in a 10% formalin solution. Each segment was cut into proximal, middle and distal stent segments for histomorphometric analysis. Tissue specimens were embedded in a cold-polymerizing resin (Technovit 7100, Heraus Kulzer GmbH, Wehrheim, Germany). Sections 5 microns thick were cut with a rotary heavy-duty microtome (HM 360, Microm, Walldorf, Germany) equipped with a hard metal knife and stained with hematoxylin-eosin, elastic stain and with phosphotungstic acid hematoxylin stain. Examination was performed using a light microscope by an experienced pathologist, who was blinded to the type of stent inspected. Injury of the arterial wall due to stent deployment (and eventually inflammation induced by the polymer) was evaluated for each stent filament and graded as described by Schwartz et al. (J Am Coll Cardiol 1992; 19(2):267-74).
Grade 0=internal elastic membrane intact, media compressed but not lacerated;
Grade 1=internal elastic membrane lacerated; Grade 2=media visibly lacerated; external elastic membrane compressed but intact; Grade 3=large laceration of the media extending through the external elastic membrane or stent filament residing in the adventitia.
   Inflammatory reaction at each stent filament was carefully examined, searching for inflammatory cells, and scored as follows:
1=sparsely located histolymphocytes surrounding the stent filament; 2=more densely located histolymphocytes covering the stent filament, but no lymphogranuloma and/or giant cells formation found; 3=diffusely located histolymphocytes, lymphogranuloma and/or giant cells, also invading the media.
   The mean score for each stent was calculated by summing the score for each filament and dividing by the number of filaments present.

Morphometric analysis of the coronary segments harvested was performed using a computerized morphometry program (Leitz CBA 8000). Measurements of lumen area, lumen area inside the internal elastic lamina, and lumen inside the external elastic lamina were performed. In addition, the areas of stenosis and neointimal hyperplasia were calculated.

Statistics For comparison among different groups, non-paired t-test was used. Data are presented as mean value±SD. A p value≦0.05 was considered as statistically significant.

Results

Quantitative Coronary Angiography As the number of stents used for the acute study was limited, the acute study stents were grouped with those from the chronic study to evaluate the degree of over-sizing that occurred. Angiographic measurements showed that the selected arterial segments and recoil ratio of TEMPO coated groups were similar to those for the bare control group (Table 4 below). The balloon size of the 0% TEMPO Gamma, 50% TEMPO ETO, and 100% TEMPO+Top Layer ETO groups was significantly lower than the balloon size for the bare stent groups. However, no significant difference in over-sizing was observed in different groups as compared to the bare stent groups.

TABLE 4
Quantitative coronary angiography

| | | | | | Recoil | |
|---|---|---|---|---|---|---|
| | | Prestenting | Balloon size | Poststenting | ratio** | Over-sizing |
| | N | (mm) | (mm) | (mm) | (%) | (%) |
| Bare stent | 9 | 2.63 ± 0.30 | 3.17 ± 0.26 | 3.09 ± 0.28 | 2.51 ± 2.34 | 21.26 ± 9.00 |
| 0% TEMPO Gamma | 10 | 2.59 ± 0.13 | 2.96 ± 0.06* | 2.88 ± 0.09 | 2.91 ± 2.05 | 14.60 ± 4.73 |
| 50% TEMPO Gamma | 9 | 2.66 ± 0.23 | 3.02 ± 0.11 | 2.92 ± 0.14 | 3.37 ± 1.83 | 14.31 ± 6.91 |
| 0% TEMPO ETO | 9 | 2.47 ± 0.16 | 2.97 ± 0.07 | 2.86 ± 0.06* | 3.76 ± 1.89 | 20.43 ± 2.65 |
| 50% TEMPO ETO | 8 | 2.52 ± 0.14 | 2.95 ± 0.12* | 2.84 ± 0.14* | 3.87 ± 3.56 | 17.22 ± 3.72 |
| 100% TEMPO + Top | 9 | 2.42 ± 0.12 | 2.93 ± 0.10* | 2.84 ± 0.10* | 3.02 ± 2.32 | 21.02 ± 5.72 |
| Layer ETO | | | | | | |

*Comparing to bare stent group, P < 0.05
**Recoil ratio = (1 − minimal lumen diameter immediately after implantation/maximal balloon diameter) × 100(%)

Histopathology At 5 days follow-up, residual polymer material was detected around the stent filaments. The inflammatory response of all TEMPO coated stents and bare stents was low: (0% TEMPO Gamma, 1.00±0.00; 50% TEMPO Gamma, 1.00±0.00; 0% TEMPO ETO, 1.06±0.10; 50% TEMPO ETO, 1.00±0.00; and 100% TEMPO+Top Layer ETO, 1.00±0.00 compared with bare stents (1.03±0.07). A few inflammatory cells were seen adjacent to the stent filaments. Stent struts with moderate inflammatory reaction were rare. A thin thrombotic meshwork covering the stent filaments was observed. Internal elastic lamina membrane was beneath the stent filaments and the media was moderately compressed. Arterial injury caused by stent deployment was low and identical for the groups (0% TEMPO Gamma, 0.24±0.10; 50% TEMPO Gamma, 0.32±0.18; 0% TEMPO ETO, 0.28±0.01; 50% TEMPO ETO, 0.25±0.01; 100% TEMPO+Top Layer ETO, 0.13±0.08; and bare stents, 0.19±0.13).

At 6 weeks follow-up, disruption of internal elastic lamina was often seen in the bare stent group. In some sections, a few stent struts lacerated external elastic lamina and even penetrated into the adventitia. In the TEMPO coated stent groups, stent struts compressed the arterial medial layer. Some internal elastic lamina was lacerated. Only a few sections showed a disruption of arterial media and/or external elastic lamina. Compared to bare stent group, the mean injury scores of the TEMPO coated stent groups were decreased (Table 2). Furthermore, the TEMPO coated stent groups showed only a mild inflammatory response. Spare inflammatory cells were observed around the stent struts. Several stent struts showed a moderate inflammatory response. No inflammatory cells were found infiltrated into media. The mean inflammatory scores of 0% TEMPO GAMMA, 50% TEMPO GAMMA and 50% TEMPO ETO groups were significantly lower than for the bare stent group.

Morphometry

At 6 weeks follow-up (as shown in Table 5 below), the lumen area of 100% TEMPO+Top Layer ETO was the smallest among the groups. Compared to the lumen area of the bare stent group, however, no significant difference was observed (4.29±2.28 vs 3.60±0.99, P>0.05). The neointimal hyperplasia and area stenosis of all TEMPO groups were lower than those for the bare stent group, but only the 0% TEMPO Gamma and the 50% TEMPO Gamma groups showed a significant decrease in neointimal hyperplasia and area stenosis. The neointimal hyperplasia of the 50% TEMPO Gamma group was the lowest.

TABLE 5
Histomorphometric analysis of stented vessel segments at 6 weeks follow-up

| | | Lumen | Neointimal | Area | | |
|---|---|---|---|---|---|---|
| | | Area | Hyperplasia | Stenosis | Inflammation | |
| | N | (mm²) | (mm²) | (%) | Score | Injury Score |
| Bare stent | 24 | 4.29 ± 2.28 | 1.78 ± 0.79 | 35 ± 23 | 1.09 ± 0.14 | 0.62 ± 0.46 |
| 0% TEMPO | 24 | 4.45 ± 0.90 | 1.26 ± 0.41* | 23 ± 9* | 1.02 ± 0.05* | 0.34 ± 0.18** |
| Gamma | | | | | | |
| 50% TEMPO | 24 | 4.31 ± 0.70 | 1.10 ± 0.18** | 21 ± 4* | 1.02 ± 0.05* | 0.39 ± 0.27* |
| Gamma | | | | | | |
| 0% TEMPO ETO | 24 | 4.15 ± 0.82 | 1.42 ± 0.61 | 26 ± 11 | 1.03 ± 0.07 | 0.31 ± 0.24** |
| 50% TEMPO ETO | 24 | 4.03 ± 0.78 | 1.36 ± 0.51 | 26 ± 10 | 1.01 ± 0.04* | 0.30 ± 0.18** |
| 100% TEMPO + Top | 24 | 3.60 ± 0.99 | 1.47 ± 0.68 | 30 ± 14 | 1.04 ± 0.07 | 0.46 ± 0.26 |
| Layer ETO | | | | | | |
| Bare stent | 24 | 4.29 ± 2.28 | 1.78 ± 0.79 | 35 ± 23 | 1.09 ± 0.14 | 0.62 ± 0.46 |
| 0% TEMPO | 48 | 4.24 ± 0.91 | 1.41 ± 0.61* | 25 ± 11* | 1.03 ± 0.06* | 0.33 ± 0.21** |
| 50% TEMPO | 48 | 4.13 ± 0.69 | 1.27 ± 0.42** | 24 ± 8** | 1.02 ± 0.05** | 0.34 ± 0.23** |
| 100% TEMPO + Top | 24 | 3.60 ± 0.99 | 1.47 ± 0.68 | 30 ± 14 | 1.04 ± 0.07 | 0.46 ± 0.26 |
| Layer ETO | | | | | | |

Comparing to bare stent group, *= P < 0.05, **= P < 0.01

### CONCLUSION

The TEMPO coated and bare stents elicited a similar tissue response at 5 days follow-up. No additional inflammatory response or increased thrombus formation was observed for the TEMPO coated stents at that time point. At 6 weeks follow-up, the neointimal formation induced by the TEMPO coated stent groups was lower than for the bare stent group. Both area stenosis and neointimal hyperplasia of 0% TEMPO Gamma and 50% TEMPO Gamma-coated stents were significantly lower than for the bare stent group. In addition, a significantly decreased peri-strut inflammation for the 0% TEMPO GAMMA, 50% TEMPO GAMMA and 50% TEMPO ETO-coated stents was observed as compared to the bare stent group. In conclusion, The TEMPO coating did not induce an increased tissue response. TEMPO coated stents sterilized with Gamma radiation showed a beneficial effect on neointimal formation at 6 weeks follow-up, especially in the 50% TEMPO group. Increased TEMPO loaded concentrations or/and addition of a top layer of de-protected polyester amide polymer—PEA(H)—did not show a consistent inhibitory effect on neointimal hyperplasia and area stenosis.

### Example 5

Noblesse Clinical Trial

Study Design

The Noblesse (Nitric Oxide through Biodegradable Layer Elective Study for Safety and Efficacy) Clinical Trial was conducted in human patients to determine the effects of implantation in a human of a functionalized polymer coating on a coronary stent without the presence of a drug. The stent used was the Genic stainless steel stent structure (Blue Medical Devices, BV, Helmund, the Netherlands) coated with PEA-Tempo, (Poly(Ester)Amide—4 amine Tempo) functionalized polymer (MediVas LLC, San Diego, Calif.).

The clinical trial was a multi-center, prospective, non-randomized study of forty-five patients that included angiographic follow-up at four months and angiographic and IVUS follow-up at twelve months. The study took place in three locations: Cordoba, Argentina, Curitiba, Brazil and Eindhoven, the Netherlands.

All patients were provided with a written informed consent prior to enrollment in the study. Patients were required to have stable or unstable angina pectoris or a positive exercise test, be at least eighteen years old, have a single, de-novo target lesion in native coronary artery, have the reference vessel be visually estimated to be greater than 2.75 mm and less than 3.50 mm in diameter, have target lesion stenosis greater than 50% and less than 100%, and have a target lesion less than 15 mm in length.

The primary endpoint of the study was the late loss of the luminal area at four months and twelve months after stent placement. Secondary endpoints were 30 day, 60 day, 120 day, and 12 month MACE (major arterial coronary event), death, recurrent myocardial infarction, or target lesion revascularization (requiring re-stenting).

Prior to the implantation procedure, each patient received at least 100 mg aspirin before stenting and oral clopidogrel of 300 mg before PTCA. Each patient received intracoronary nitroglycerin of 50-200 μg prior to baseline angiography, during post-stent deployment and after final post dilatation angiography. Each patient also received sufficient heparin to maintain ACT of 250-300 seconds. For 28 days after the procedure each patient received 75 mg/d of Clopidogrel.

Patient Demographics Of the forty-five patients, thirty-one (69%) were male. The patients ranged in age from 38 to 83 years, with a mean age of 62 years. Twenty-two patients were enrolled in Brazil, eighteen in Argentina, and five in the Netherlands.

Lesion Characteristics The vessel in the heart treated in patients

| | |
|---|---|
| Right coronary artery | 40.0% |
| Left anterior descending artery | 7.5% |
| Left circumflex artery | 22.5% |
| AHA/ACC class^{a} | |
| A: | 14.3% |
| B1: | 61.9% |
| B2: | 23.8% |
| TIMI 3 (a blood flow measure)^{b} | 100% |
| Angulation > 45%^{c} | 19.1% |
| Moderate vessel tortuousity^{d} | 23.8% |
| Avg. Ref Vessel Diameter:^{e} | 2.98 ± 0.32 mm |
| Avg. Minimum luminal diameter prior to stenting:^{f} | 1.05 ± 0.34 mm |
| Avg.Minimum luminal diameter 4 mos. after stenting:^{g} | 2.74 ± 0.26 mm |
| Avg. Diameter of Stenosis prior to stenting:^{h} | 64.69 ± 11.59% |
| Avg. Diameter Stenosis 4 mos. after stenting:ⁱ | 8.70 ± 4.52% |
| Avg. Acute Gain^{j} | 1.69 ± 0.42 mm |

All patients were discharged 24 hours after the procedure
with no complications.

| | |
|---|---|
| Cardiac death | 0 |
| Q-wave MI (as read by electrocardiogram)^{k} | 0 |
| Non Q-wave MI | 0 |
| CABG required^{l} | 0 |
| TLR* | 0 |

At the twelve month follow-up patient results were as follows:

| | |
|---|---|
| Cardiac death | 0 |
| Q-wave MI (as read by electrocardiogram) | 0 |
| Non Q-wave MI | 0 |
| Coronary artery bypass surgery required | 0 |
| TLR^{m} | 1 |
| Average minimum luminal diameter at 12 months | 2.87 ± 0.31 mm |
| post stenting:: | |

^{a}The AHA/ACC class refers to the American Heart Association/American College of Cardiology rating system for severity of blockage. The severity increases from mild (A1) through moderate (B1) to severe (B2). Total occlusion is C.
^{b}TIMI 3 refers to thrombolysis in myocardial infarction. These are a rating of the blood's ability to flow, going from 1 to 3, with 3 being the most flow (or least likely to have thrombosis). TIMI 4 is total occlusion.
^{c}Angulation > 45% means the percentage of target arteries that have a bend of 45% or more within the target lesion.
^{d}Moderate vessel tortuousity (slide 5) is an objective evaluation by the interventionalist as to the degree of “twistiness” of the artery.
^{e}Ref Vessel Diameter is the size of the native artery immediately proximal to the target lesion.
^{f}MLD Pre (means “minimum luminal diameter” and describes the smallest cross section of the artery at the lesion site prior to stent placement.
^{g}MDL Post means “minimum luminal diameter” and describes the smallest cross section of the artery at the lesion site after stent placement.
^{h}Diameter Stenosis Pre is calculated by subtracting MLD Pre from Ref Vessel Diameter and dividing by Ref Vessel Diameter.
ⁱDiameter Stenosis Post is calculated by subtracting MLD Post from Ref Vessel Diameter and dividing by Ref Vessel Diameter.
^{j}Acute gain is Diameter Stenosis Pre-subtracted from Diameter Stenosis Post.
^{k}Q-wave MI and Non Q-wave MI are two forms of myocardial infractions (heart attacks) as indicated by electrocardiogram.
^{l}CABG is coronary artery bypass graph and refers to bypass surgery.
^{m}TLR is total lesion revascularization and refers to the placement of a second stent to correct the failure of the first stent.

Conclusions The PEA-4 Amine Tempo polymer was shown to be a safe form of biodegradable, biocompatible polymer and the polymer alone, without added drug, demonstrated a unique capability to preserve and even enhance the beneficial effect of the invention stents in coronary arteries as measured by the increase in average minimum luminal diameter in treated heart arteries 12 months after stent emplacement.

### Example 6

Cell Recruitment to Bioactive Agents To select appropriate bioligands for use as recruitment factors in wound healing stent applications, an in vitro adhesion assay was developed. This assay can distinguish between endothelial cells (ECs) and smooth muscle cells (SMCs) to aid in selecting potential attachment factors. Both the ECs and SMCs used in these assays were purchased from Cambrex (Baltimore, Md.) (HASMC=Human Aortic Smooth Muscle Cells and HCAEC=Human Coronary Artery Endothelial Cells).

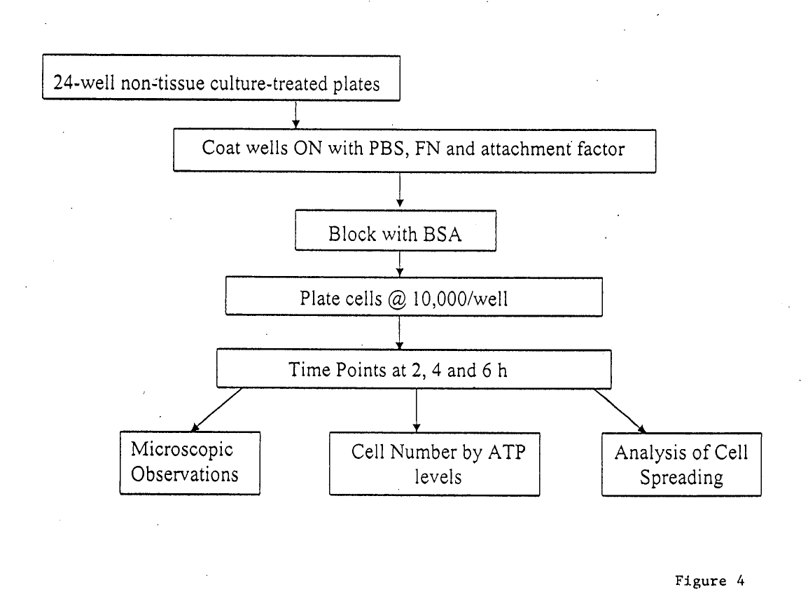

FIG. 4 shows the flow chart of the protocol followed for this assay. The attachment factor, in a phosphate buffered saline (PBS) solution, was coated onto a non-tissue culture dish and allowed to adsorb overnight at 4° C. The following day the plate was blocked for 1 hour at room temperature with heat-inactivated, 0.2% bovine serum albumin (BSA) solution (in PBS) to prevent non-specific attachment. A timed adhesion assay was then conducted. The assay includes negative control wells coated only with PBS and positive control wells coated with fibronectin. So far, none of the adhesion factors tested has surpassed the cell adhesion and cell spreading induced by fibronectin. In addition to adhesion, spreading is also an important consideration in determining the suitability of a substrate. If the cells are not able to spread, it is unlikely that the cells will proliferate on that surface.

Initial efforts focused on potential recruitment factors with low affinity but present in high density. A variety of potential recruitment factors were tested, including:
1. Sialyl Lewis X, a ligand for Selectin receptors found on endothelium;
2. CS5, whose amino acid sequence is Gly-Glu-Glu-Ile-Gln-Ile-Gly-His-Ile-Pro-Arg-Glu-Asp-Val-Asp-Tyr-His-Leu-Tyr-Pro (SEQ ID NO:9). CS5 is found in the Type III connecting segment of fibronectin, an extracellular matrix protein known to bind many different cells, including ECs. The sequence for the CS5 peptide contains the amino acid sequence REDVDY (underlined) (SEQ ID NO:10); and
3. GREDVDY (SEQ ID NO:11), which includes a G linker placed on the REDVDY sequence.)

Of the bioligands tested to date, CS5 and GREDVDY gave the most promising adhesion data with the best sites for conjugation to the polymers used in making the invention stents. Even though neither of these peptide sequences equaled the large molecule fibronectin in cell adhesion or spreading, surprisingly both peptide sequences showed specificity for ECs over SMCs and these small peptide sequences can be readily synthesized and bound to the polymers used in the polymers used in manufacture of the invention stents and implantable medical device coverings.

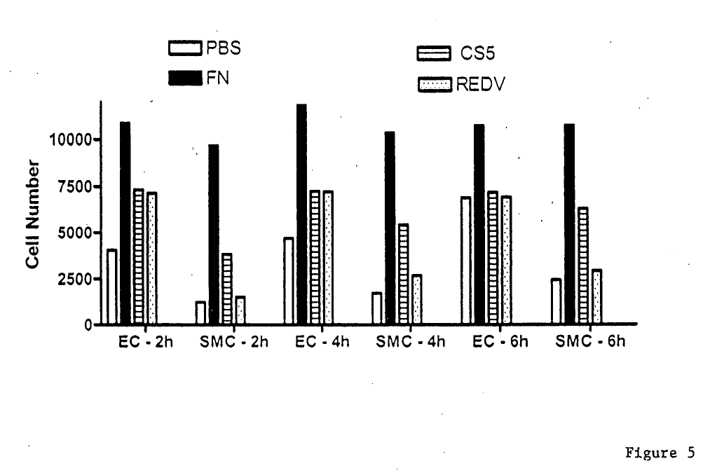

In addition to microscopic observations, cell adhesion was quantitated using an ATP assay. Data of a representative adhesion assay quantitation by ATP standard curve is shown in the graph in FIG. 5, which illustrates the comparative results obtained at 2, 4 and 6 hours into the assay. The assay can identify the number of cells that are adhered to a specific substrate; however, it does not take into consideration cell spreading. The cell spreading determined in microscopic observations may indicate that cell spreading can increase the overall degree of cell adhesion since more space is occupied by a well spread cell than by an adhered cell that has not spread on the surface, due to timing of data points or appropriateness of the substrate used. The ATP data are useful to support the observational findings of the adhesion assay but cannot replace the adhesion assay.

### Example 7

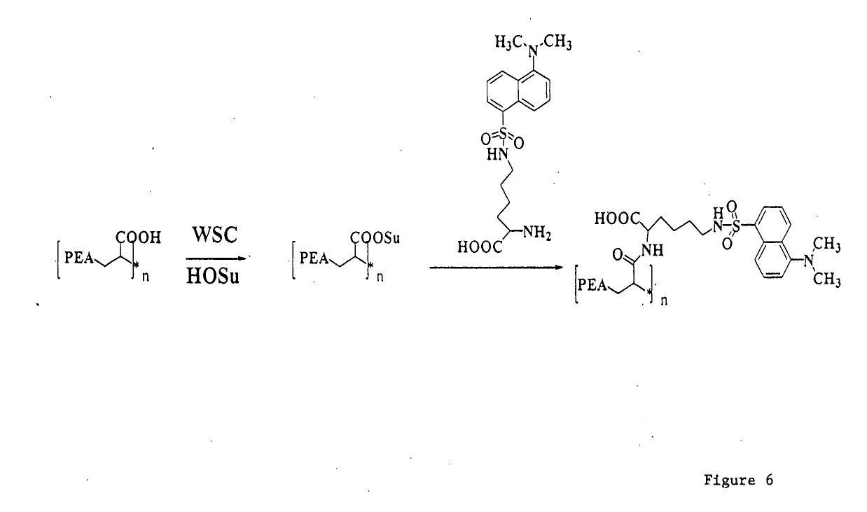

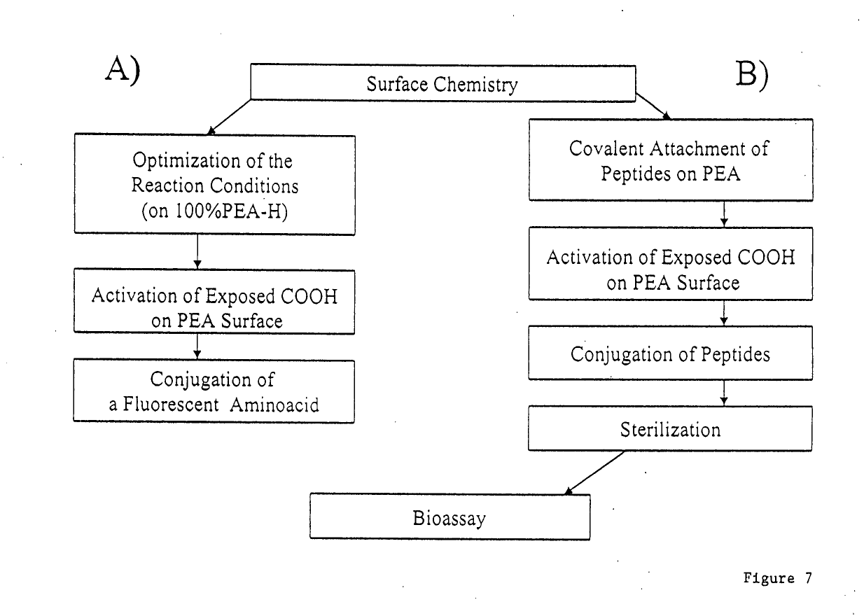

Cell Recruitment to Bioactive Agent-Polymer Conjugates Based upon the promising results from the adhesion assays, the next step was to conjugate the most effective of the identified recruitment factors to the stent polymer to assess the increased adhesion to the polymer induced with these potential recruitment factors. The first conjugation was done to the PEA-H version of the polymer (acid) since this polymer has suitable sites for conjugation. The peptides can be covalently bound to this polymer via a wide variety of suitable functional groups. For example, when the biodegradable polymer is a poly(ester amide) (PEA) containing Lysine residues, the carboxyl groups from the Lysine residues can be used to react with a complementary moiety on the peptide, such as an hydroxy, amino, thio moiety, and the like (5). Specifically, the PEA-H polymer with free COOH reacts with water soluble carbodiimide (WSC) and N-Hydroxysuccinimide (HOSu) to produce an activated ester, which, in turn, reacts with an amino functional group of a peptide to provide an amide linkage (FIG. 7B). By using a fluorescent dansyl-lysine (FIG. 6), the optimal reaction conditions for activation and conjugation were determined (FIG. 7A).

The conjugation of CS5 and GREDVDY peptides to the polymer was then performed using the same protocol (FIG. 7B). The adhesion assay showed that the conjugation of the peptides did not alter their ability to bind to cells; and, further, that the ECs when compared to the SMCs adhered significantly better to the conjugated peptides than on the unconjugated PEA-H polymer.

A similar protocol (see flow chart FIG. 7B) was used to conjugate combinations of the acid polymer with PEA polymer of structure (I) containing acetylated ends and benzylated COOH groups, (PEA-AcBz) and PEA-TEMPO (50/50 and 10/90), respectively. By combining the conjugatable acid form with the other polymers, a determination could be made whether the presence of the recruitment peptide on the polymer conferred an advantage in EC recruitment.

Microscopic observations taken at 2 h, 4 h and 6 h from duplicate wells from two representative adhesion assays are summarized in Table 6 below.

TABLE 6
Summary of Assays with Conjugated Peptides on Polymer

| | 50/50 H/Bz 2a | 50/50 H/Bz 2a | 10/90 H/Bz 2a | 10/90 H/Bz | | |
|---|---|---|---|---|---|---|
| | & 2b | & 2b | & 2b | 2a & 2b | | |
| | 50/50 H/T 3a | 50/50 H/T 3a | 10/90 H/T 3a | 10/90 H/T | | |
| | & 3b | & 3b | & 3b | 3a & 3b | Plastic | Plastic |
| Coating/Conj | Assay 1 | Assay 2 | Assay 1 | Assay 2 | Assay 1 | Assay 2 |
| 2 h | | | | | | |
| 2A | | | | | | |
| PBS | 20% r | 20-30% r/s | 30% r/s | 30% r/s | 20% r/s | 20-30% |
| | | | | | | r/s/sp |
| Conj CS5 | 20% r | 20-30% r | 30% r/s | 30% r/s | | |
| 2B | | | | | | |
| PBS | 20% | 30% r/s | 30% r | 30% r/s | 20-30% r | 30% r/s |
| Conj REDV | 20-30% r | 30-40% r/s/sp | 30% r/s | 30% r/s/sp | | |
| 3A | | | | | | |
| PBS | 30% r | 20-30% r/s | 30% r/s | 30% r/s | 20-30% r | 20-30% |
| | | | | | | r/s |
| Conj CS5 | 20-30% r/s | 30% r/s | 30% r/s | 30% r/s/sp | | |
| 3B | | | | | | |
| PBS | 20-30% r | 30% r/s/sp | 20-30% r/s | 30% r/s/sp | 20-30% r | 20-30% |
| | | | | | | r/s |
| Conj REDV | 20-30% r | 30% r/s/sp | 30% r/s | 30% r/s/sp | | |
| 4 h | | | | | | |
| 2A | | | | | | |
| PBS | 30% r | 20-30% r | 40% r/s | 30% r/s/sp | 30% r/s | |
| Conj CS5 | 30% r | 30% r/s/sp | 40% r/sp | 30% s/sp | | |
| 2B | | | | | | |
| PBS | 30% r | 30-40% r/s | 30% r/s | 30-40% | 20% r | 30% s/sp |
| | | | | r/s/sp | | |
| Conj REDV | 30% s/sp | 30-40% s/sp | 30-40% r/s/sp | 30-40% s/sp | | |
| 3A | | | | | | |
| PBS | 30% r | 30% r/s | 30% r/s | 30% s/sp | 30% r | 30% r/s |
| Conj CS5 | 30% r | 30-40% r/s/sp | 30% r/s/sp | 30-40% s/sp | | |
| 3B | | | | | | |
| PBS | 30% r | 30% r/s/sp | 30% r/s/sp | 30% s/sp | 30% r | 20-30% |
| | | | | | | r/s/sp |
| Conj REDV | 30% | 30% r/s/sp | 30-40% s/sp | 40% s/sp | | |
| 6 h | | | | | | |
| 2A | | | | | | |
| PBS | 20% r | 20% r/s | 30% r/s | 30% r/s/sp | 20% r/s | 30% r/s/sp |
| Conj CS5 | 20% r | 30% r/s | 30-40% s/sp | 30% r/s/sp | | |
| 2B | | | | | | |
| PBS | 20% r | 30% r/s/sp | 30% r/s/sp | 30-40% | 20% r | 30% r/s/sp |
| | | | | r/s/sp | | |
| Conj REDV | 30% r/s | 30-40% r/s/sp | 30% s/sp | 30-40% | | |
| | | | | r/s/sp | | |
| 3A | | | | | | |
| PBS | 20% r | 30% r/s | 30% r/s/sp | 30-40% | 20% r | 30-40% |
| | | | | r/s/sp | | r/s |
| Conj CS5 | 20% r | 30% r/s | 30-40% r/s/sp | 30% r/s/sp | | |
| 3B | | | | | | |
| PBS | 20% r | 30% r/s | 30% r | 30-40% s/sp | 20% r | 30% r/s |
| Conj REDV | 20% r | 30-40% s/sp | 30-40% r/s/sp | 40% s/sp | | |

r = round,
s = spindly,
sp = spread;
50/50 H/Bz = 50% PEA-H and 50% PEA-Ac-Bz;
10/90 H/Bz = 10% PEA-H and 90% PEA-Ac-Bz;
50/50 H/T = 50% PEA-H and 50% PEA-Ac-TEMPO;
10/90 H/T = 10% PEA-H and 90% PEA-Ac-TEMPO.

A complete evaluation of the assays with conjugated peptides on the polymer (Table 2), showed a benefit to the presence of the recruitment peptides on the polymer. The following combinations of polymer conjugated to the GREDVDY peptide resulted in an increased adhesion over basal levels in both assays 1 and 2 (early and late time points). 50/50 PEA-H/PEA-Ac-Bz (H/Bz) and 10/90 PEA-H/PEA-TEMPO(H/T) conjugated to GREDVDY—at middle and late time points. Surprisingly, the shorter peptide (7 mer) proved more bust in cell recruitment than the longer (20 mer) CS5 peptide.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

Although the invention has been described with reference to the above examples, it will be understood that modifications and variations are encompassed within the spirit and scope of the invention. Accordingly, the invention is limited only by the following claims.

What is claimed is:

1. A wound-healing composition comprising at least one wound healing agent dispersed in biodegradable, biocompatible polymer,
wherein the polymer is a poly(ester amide) (PEA) having a structural formula described by structural formula (I), 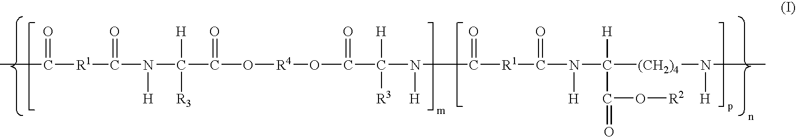
and wherein n ranges from about 5 to about 150, m ranges about 0.1 to about 0.9: p ranges from about 0.9 to about 0.1; wherein R¹ is selected from the group consisting of (C₂-C₂₀) alkylene or (C₂-C₂₀) alkenylene; R² is hydrogen or (C₆-C₁₀)aryl (C₁-C₆) alkyl or a protecting group; R³ is selected from the group consisting of hydrogen, (C₁-C₆) alkyl, (C₂-C₆) alkenyl, (C₂-C₆) alkynyl and (C₆-C₁₀)aryl(C₁-C₆) alkyl; and R⁴ is selected from the group consisting of (C₂-C₂₀) alkylene, (C₂-C₂₀) alkenylene or alkyloxy, and bicyclic-fragments of 1,4:3,6-dianhydrohexitols of general formula (II): 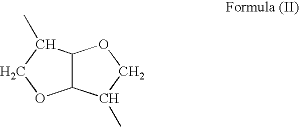
except that for unsaturated polymers having the chemical structure of structural formula (I), R¹ and R⁴ are selected from (C₂-C₂₀) alkylene and (C₂-C₂₀) alkenylene; wherein at least one of R¹ and R⁴ is (C₂-C₂₀) alkenylene; n is about 5 to about 150; each R² is independently hydrogen, or (C₆-C₁₀)aryl(C₁-C₆)alkyl; and each R³ is independently hydrogen, (C₁-C₆)alkyl, (C₂-C₆)alkenyl, (C₂-C₆)alkynyl, or (C₆-C₁₀)aryl(C₁-C₆)alkyl;
or a poly(ester urethane) (PEUR) having a chemical formula described by general structural formula (III), 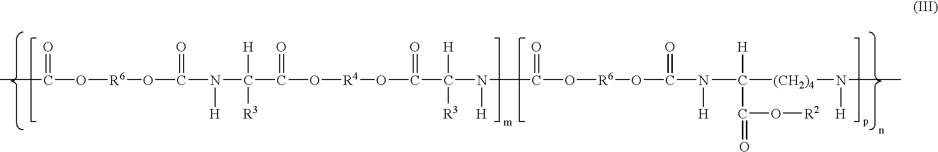
and wherein n ranges from about 5 to about 150, m ranges about 0.1 to about 0.9: p ranges from about 0.9 to about 0.1; wherein R² is hydrogen or (C₆-C₁₀)aryl(C₁-C₆) alkyl or a protecting group; R³ is selected from the group consisting of hydrogen, (C₁-C₆) alkyl, (C₂-C₆) alkenyl, (C₂-C₆) alkynyl and (C₆-C₁₀) aryl(C₁-C₆) alkyl; R⁴ is selected from the group consisting of (C₂-C₂₀) alkylene, (C₂-C₂₀) alkenylene or alkyloxy, and bicyclic-fragments of 1,4:3,6-dianhydrohexitols of general formula (II); and R⁶ is independently selected from (C₂-C₂₀) alkylene, (C₂-C₂₀) alkenylene or alkyloxy, and bicyclic-fragments of 1,4:3,6-dianhydrohexitols of general formula (II).

2. The composition of claim 2, wherein R³ is CH₂Ph.

3. The composition of claim 2 wherein

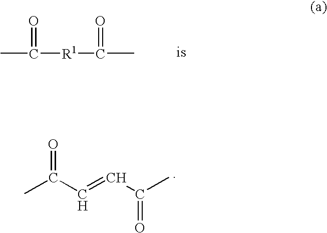

4. The composition of claim 3, wherein R⁴ is selected from —CHCH═CH—CH₂—, —(CH₂)₄—, and —(CH₂)₆—.

5. The composition of claim 2, wherein R⁴ is —CH₂—CH═CH—CH₂—.

6. The composition of claim 1, wherein the composition is implantable.

7. The composition of claim 1, wherein the composition further comprises a hydrogel and the wound healing agent is additionally dispersed within the hydrogel.

8. The composition of claim 7, wherein the hydrogel is derived from both hydrophobic and hydrophilic components and has a one-phase crosslinked polymer network structure.

9. The composition of claim 1, wherein the composition is formulated as a wound dressing.

10. The composition of claim 9, wherein the composition further comprises a biocompatible hydrogel, wherein the polymer and the hydrogel are in separate portions of the wound dressing and the at least one wound healing agent is dispersed in the polymer, the hydrogel, or both.

11. The composition of claim 4, wherein the separate portions are contiguous layers.

12. The composition of claim 11, further comprising an occlusive layer contiguous with either the polymer or hydrogel layer.

13. The composition of claim 10, wherein the at least one wound healing agent is released from the composition at a controlled rate as a result of biodegradation of the polymer, the hydrogel, or both.

14. The composition of claim 1, wherein the at least one wound healing agent is covalently bonded to the polymer.

15. The composition of claim 1, wherein the wound healing agent is a wound healing cell selected from a pericyte, endothelial cell, progenitor endothelial cell or combination thereof dispersed in the hydrogel, and the composition further comprises a growth medium for the cell imbibed in the hydrogel.

16. The composition of claim 1, wherein the bioactive agent is an antibody or molecular ligand that specifically binds to a molecule selected from Intercellular adhesion molecules (ICAMs; Vascular cell adhesion molecules (VCAMs), Neural cell adhesion molecules (NCAMs); Platelet endothelial cell adhesion molecules (PECAMs); or Leukocyte-endothelial cell adhesion molecules (ELAMs).

17. The composition of claim 1, wherein the composition is formulated as a wound dressing and the wound healing agent is a tissue graft material supported by the polymer.

18. The composition of claim 1, wherein wound healing agent is an extra-cellular matrix protein selected from a glycosaminoglycan, a proteoglycan, collagen; elastin; fibronectin, laminin, alginate, a chitin derivative, and a combination thereof.

19. The composition of claim 1, wherein the wound healing agent is a proteinaceous growth factor selected from Platelet Derived Growth Factor-BB (PDGF-BB), Tumor Necrosis Factor-alpha (TNF-alpha), Epidermal Growth Factor (EGF), Keratinocyte Growth Factor (KGF), Thymosin B4, Regranex®, Procuren®, and combinations thereof.

20. The composition of claim 1, wherein the wound healing agent is a proteinaceous growth factor is selected from vascular Endothelial Growth Factors (VEGFs), Fibroblast Growth Factors (FGFs), Tumor Necrosis Factor-beta (TNF-beta), and Insulin-like Growth Factor-1 (IGF-1).

21. The composition of claim 1, wherein the wound healing agent is an anti-proliferant agent.

22. The composition of claim 21, wherein the anti-proliferant agent is selected from a rapamycin, paclitaxel, Sirolimus, Everolimus, or tacrolimus.

23. The composition of claim 1, wherein the wound healing agent is selected from simvastatin, atorvastatin, fluvastatin, pravastatin, lovastatin, rosuvastatin, 17-allylamino-17-demethoxygeldanamycin (17AAG); Epothilone D, 17-dimethylaminoethylamino-17-demethoxy-geldanamycin or Cilostazol

24. The composition claim 1, wherein the wound healing agent is selected from vitamin A and synthetic inhibitors of lipid peroxidation.

25. The composition of claim 1, wherein the at least one wound healing agent is selected from arginine, lysine, aminoxyls, furoxans, nitrosothiols, nitrates, anthocyanins, sphingosine-1-phosphate, or lysophosphatidic acid.

26. The composition of claim 1, wherein the polymer is in the form of a sheet, pad, or mat.

27. The composition of claim 1, wherein the polymer is in the form of a coating on at least a portion of an implantable surgical device.

28. The composition of claim 1, wherein the implantable surgical device is an implantable cardiovascular or orthopedic device.

29. The composition of claim 28, wherein the surgical device is a porous cardiovascular stent.

30. The composition of claim 29, wherein the at least one wound healing agent is a ligand that promotes re-endothelialization of endothelial cells

31. The composition of claim 1, wherein the wound healing agent is attached to the biodegradable polymer via a linker.

32. The composition of claim 1, wherein the wound healing agent is released from the composition under in vivo conditions over a time selected from about twenty-four hours, about seven days, about thirty days, or about ninety days.

33. A method for promoting natural healing of a wound comprising contacting the wound with a composition of claim 1 under conditions suitable for promoting natural healing of the wound.

34. The method of claim 33, wherein the wound is a chronic wound.

35. The method of claim 33, wherein the method further comprises placing the polymer in contact with a wound bed and allowing the polymer to biodegrade, releasing the wound healing agent into the wound bed.

36. The method of claim 33, wherein the method further comprises placing the biodegradable hydrogel in contact with the wound bed and allowing the polymer to biodegrade, releasing the wound healing agent into the wound bed.

37. The method of claim 33, wherein the wound is a venous stasis ulcer, diabetic ulcer, pressure ulcer, or ischemic ulcer.

38. The method of claim 33, wherein the natural healing comprises re-endothelialization of the wound bed.

39. A multilayer bioactive wound dressing comprising:
a non-stick layer comprising a biodegradable hydrogel;
a supporting layer comprising a biodegradable polymer, wherein the supporting layer overlies the non-stick layer; and
at least one wound healing agent that produces a wound healing effect in situ dispersed within the polymer, the hydrogel, or both,
wherein the polymer is a PEA having a structural formula described by structural formula (I), 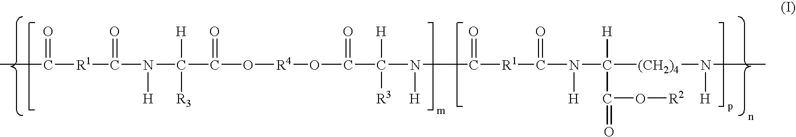
and wherein n ranges from about 5 to about 150, m ranges about 0.1 to about 0.9: p ranges from about 0.9 to about 0.1; wherein R¹ is selected from the group consisting of (C₂-C₂₀) alkylene or (C₂-C₂₀) alkenylene; R² is hydrogen or (C₆-C₁₀)aryl (C₁-C₆) alkyl or a protecting group; R³ is selected from the group consisting of hydrogen, (C₁-C₆) alkyl, (C₂-C₆) alkenyl, (C₂-C₆) alkynyl and (C₆-C₁₀)aryl(C₁-C₆) alkyl; and R⁴ is selected from the group consisting of (C₂-C₂₀) alkylene, (C₂-C₂₀) alkenylene or alkyloxy, and bicyclic-fragments of 1,4:3,6-dianhydrohexitols of general formula (II): 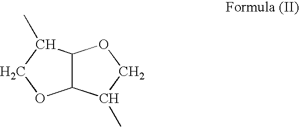
except that for unsaturated polymers having the chemical structure of structural formula (I), R¹ and R⁴ are selected from (C₂-C₂₀) alkylene and (C₂-C₂₀) alkenylene; wherein at least one of R¹ and R⁴ is (C₂-C₂₀) alkenylene; n is about 5 to about 150; each R² is independently hydrogen, or (C₆-C₁₀)aryl(C₁-C₆)alkyl; and each R³ is independently hydrogen, (C₁-C₆)alkyl, (C₂-C₆)alkenyl, (C₂-C₆)alkynyl, or (C₆-C₁₀)aryl(C₁-C₆)alkyl;
or a PEUR having a chemical formula described by general structural formula (III), 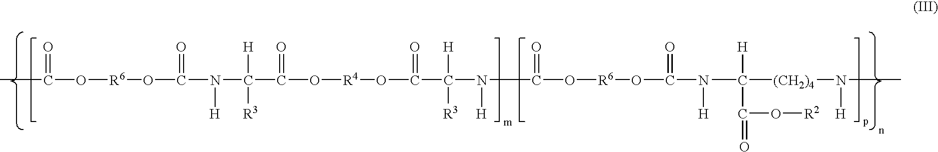
and wherein n ranges from about 5 to about 150, m ranges about 0.1 to about 0.9: p ranges from about 0.9 to about 0.1; wherein R² is hydrogen or (C₆-C₁₀)aryl(C₁-C₆) alkyl or a protecting group; R³ is selected from the group consisting of hydrogen, (C₁-C₆) alkyl, (C₂-C₆) alkenyl, (C₂-C₆) alkynyl and (C₆-C₁₀) aryl(C₁-C₆) alkyl; R⁴ is selected from the group consisting of (C₂-C₂₀) alkylene, (C₂-C₂₀) alkenylene or alkyloxy, and bicyclic-fragments of 1,4:3,6-dianhydrohexitols of general formula (II); and R⁶ is independently selected from (C₂-C₂₀) alkylene, (C₂-C₂₀) alkenylene or alkyloxy, and bicyclic-fragments of 1,4:3,6-dianhydrohexitols of general formula (II).

40. The wound dressing of claim 39, wherein R³ is CH₂Ph.

41. The wound dressing of claim 39 wherein

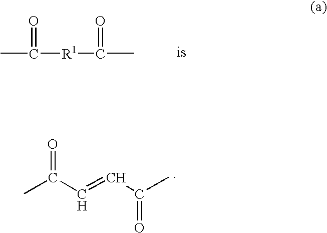

42. The wound dressing of claim 41, wherein R⁴ is selected from —CH₂—CH═CH—CH₂—, —(CH₂)₄—, and —(CH₂)₆—.

43. The wound dressing of claim 40, wherein R⁴ is —CH₂—CH═CH—CH₂—.

44. The wound dressing of claim 39, wherein the hydrogel is derived from both hydrophobic and hydrophilic components and has a one-phase crosslinked polymer network structure.

45. The wound dressing of claim 39, further comprising a tape or wrap for holding the non-stick layer against a wound.

46. The wound dressing of claim 45, wherein the wound is chronic and the tape or wrap is elastic and of sufficient length to use for applying compression to the wound.

47. The wound dressing of claim 46, wherein the chronic wound is a venous stasis ulcer, diabetic ulcer, pressure ulcer, or ischemic ulcer.

48. The wound dressing of claim 39, wherein the wound healing agent is selected from wound healing cells, tissue grafts, extra cellular matrix proteins, proteinaceous growth factors, antimicrobials, anti-inflammatory agents, healing promoters, biocompatible glycoproteins, and combinations thereof.

49. The wound dressing of claim 39, wherein the at least one wound healing agent is released at a controlled rate.

50. The wound dressing of claim 39, wherein the polymer and hydrogel are in separate contiguous layers and the wound dressing further comprises an occlusive layer.

51. A bioactive implantable stent comprising a porous stent structure; and a multilayered tubular coating encapsulating the stent structure, the multilayered coating comprising:
an outer drug-eluting biodegradable polymer layer, which sequesters an unbound drug;
an inner layer of a wound healing composition of claim 1; and
a biodegradable barrier layer lying between and in contact with the outer layer and the inner layer and which barrier layer is impermeable to the drug.

52. The stent of claim 51, wherein the at least one bioactive agent comprises a ligand that promotes re-endothelialization of endothelial cells, which bioactive agent is attached to the polymer in the inner layer.

53. The stent of claim 52, wherein the ligand is selected from peptides that promote endothelial cell growth.

54. The stent of claim 53, wherein the ligand is selected from bradykinins 1 and 2.

55. The stent of claim 51, further comprising an additional bioactive agent.

56. The stent of claim 55, wherein the additional bioactive agent is rapamycin, paclitaxel, everolimus, or a statin.

57. The stent of claim 51, wherein the polymer barrier layer comprises polyester, poly(amino acid), poly(ester amide), poly(ester urethane), polyurethane, polylactone, poly(ester ether), or copolymers thereof.

58. The stent of claim 51, wherein the stent is sized for intravascular insertion.


SEQUENCE LISTING (11 sequences)

SEQ ID NO: 1 - protein, 61 aa, Artificial sequence [sequence omitted]

SEQ ID NO: 2 - protein, 55 aa, Artificial sequence
  Small bacterial proteinaceous motif
TYKLILNGKTLKGETTTEAVDAATAEKVFKQYANDNGVDGEWTYDDATKTFTVTE

SEQ ID NO: 3 - protein, 61 aa, Artificial sequence [sequence omitted]

SEQ ID NO: 4 - protein, 55 aa, Artificial sequence; identical to SEQ ID NO: 2
  Synthetic construct

SEQ ID NO: 5 - protein, 10 aa, Artificial sequence
  Small proteinaceous motif
KRPPGFSPFR

SEQ ID NO: 6 - protein, 9 aa, Artificial sequence
  Small proteinaceous motif
KRPPGFSPF

SEQ ID NO: 7 - protein, 9 aa, Artificial sequence
  Small proteinaceous motif
RPPGFSPFR

SEQ ID NO: 8 - protein, 8 aa, Artificial sequence
  Small proteinaceous motif
RPPGFSPF

SEQ ID NO: 9 - protein, 20 aa, Artificial sequence
  Synthetic construct
GEEIQIGHIPREDVDYHLYP

SEQ ID NO: 10 - protein, 6 aa, Artificial sequence
  Synthetic construct
REDVDY

SEQ ID NO: 11 - protein, 7 aa, Artificial sequence
  Synthetic construct
GREDVDY